(19) United States
(12) Patent Application Publication
(10) Pub. No.: US 2021/0405030 A1
(43) Pub. Date: Dec. 30, 2021

(54) MEANS AND METHODS FOR DETERMINING METABOLIC ADAPTATION

(71) Applicant: EnFin GmbH, Heidelberg (DE)

(72) Inventor: Georg Gdynia, Heidelberg (DE)

(21) Appl. No.: 17/288,211

(22) PCT Filed: Oct. 24, 2019
(86) PCT No.: PCT/EP2019/079055

§ 371 Date: Apr. 23, 2021

(30) Foreign Application Priority Data
Oct. 24, 2018 (EP) 18202322.6

(51) Int. Cl. G01N 33/50; C12Q 1/48

(52) U.S. Cl. CPC G01N 33/5047; G01N 2333/91215; C12Q 1/485

(57) ABSTRACT

The present invention relates to a method of determining a metabolic adaptation of a living entity of interest to a first set of environmental conditions and to a second set of environmental conditions comprising (a) determining with a first substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₁ and t₂ after starting the determining reaction; (b) determining with a second substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₃ and t₄ after starting the determining reaction; wherein said second substrate concentration is at most twofold, preferably is about equal to or lower than, the K_{M} of said enzyme for said substrate; and (c) determining the metabolic adaptation of said living entity based on comparing at least one non-linear activity determined in step (a) and/or (b) to at least one further activity determined in step (a) and/or (b). The present invention also relates to devices and further methods related thereto; as well as to a redox-fixed HMGB1 derivative polypeptide.

The present invention relates to a method of determining a metabolic adaptation of a living entity of interest to a first set of environmental conditions and to a second set of environmental conditions comprising (a) determining with a first substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₁ and t₂ after starting the determining reaction; (b) determining with a second substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₁ and t₀ after starting the determining reaction; wherein said second substrate concentration is at most twofold, preferably is about equal to or lower than, the K_{M} of said enzyme for said substrate; and (c) determining the metabolic adaptation of said living entity based on comparing at least one non-linear activity determined in step (a) and/or (b) to at least one further activity determined in step (a) and/or (b). The present invention also relates to devices and further methods related thereto; as well as to a redox-fixed HMGB1 derivative polypeptide.

Determining the capability of living cells to adapt to specific environmental conditions is of high importance in ecology, medicine, and other fields of life science, since the ability to adapt, or not, may determine cell fate. Testing for adaptation usually comprises maintaining cells of interest under the environmental conditions to be tested, and to evaluate survival and/or proliferation. However, this proceeding may be cumbersome and its practicability may be limited in case growth conditions for the specific cell are not known. Nonetheless, determining whether cells can switch e.g. to energy production under hypoxic conditions is desirable e.g. in cancer treatment.

The enzyme pyruvate kinase M2 (PKM2) was recently identified as a specific target for cancer therapy, which is in particular produced in cancer cells. PKM2 is a pacemaker enzyme of glycolysis and occurs in two forms: the tetrameric form serves in the aerobic degradation of glucose (oxidative phosphorylation) and has a low K_{M} value for its substrate phosphoenolpyruvate (PEP); accordingly, the tetrameric form is highly active at physiological concentrations of PEP, causing channeling of glucose into energy metabolism. The dimeric form of PKM2 has a high K_{M} value for PEP and is almost inactive at physiological concentrations of PEP, causing glycolytic intermediates before pyruvate to be channeled into synthetic processes, in particular in glycolysis under conditions of limited oxygen supply.

It was also reported that central carbon metabolism in malignant cells is regulated by a change of activity of glycolytic enzymes under anoxia (compared to normoxia) and that this change correlates with an unfavourable clinical outcome (EP2821790 A1; WO2017/098051 A2; Gdynia et al. (2018), EBioMedicine 32:125). Nonetheless, tumor cell metabolism differs in many aspects from non-tumor cell metabolism (Gatenby et al. (2004), Nature Reviews Cancer 4:891; Amoedo et al. (2013), Biosci. Rep. 33:e00080).

The distinction between patients whose immune cells are not activatable by intrinsic or extrinsic stimuli normally potently activating the human immune system (e.g. DAMPs (Damage Associated Molecular Patterns), PAMPs (Pathogen Associated Molecular Patterns) (Lee et al. (20189, PNAS 115 (19):E4463; Venereau et al. (2012), J. Exp. Med. 209 (9):1519) and those who respond to these stimuli (i.e. have activatable immune cells) is a major challenge in medicine. It is in particular important in disorders where the patient's outcome is intertwined with an activatable immune system (Cheng et al. (20169, Nat Immunol., 17 (4):406; Alves-Filho et al. (2016), Front. Immunol.; doi: 10.3389/fimmu.2016.00145) like in pathogen-induced inflammation, autoimmune disease, sepsis, development, progression or recurrence of cancer, treatment of cancer by boosting the immune response (e.g. with checkpoint inhibitors), immunodeficiency disorders, exhaustion of immune cells (e.g. to prevent/predict T-cell exhaustion in cellular immune-therapy) (Cascone et al. (2018), Cell Metabolism 27: 977), tissue repair (Venereau et al. (2012), ibd.), proliferation and/or migration of immune (stem) cells within/to the wounded/ischemic tissue (e.g. blood, bone marrow, heart, coronary arteries, vessels, bones, brain, nerves, myelin sheaths) (Arts et al., (2017), J. Leukoc. Biol. 101:151; Delano et al. (2016), JCI 26:23; Wasmuth et al, (2005), Journal of Hepatology 42:195) and in the field of synthetic immunology (Roybal et al. (2017), Annu. Rev. Immunol. 35:229; Irving et al. (2017), Front. Immunol. doi: 10.3389/fimmu.2017.00267). Meanwhile it is accepted that immune cell activation is correlated with a marked change in immuno-metabolism. Glycolysis must be properly controlled for immunity and to prevent autoimmunity. Moreover, there is evidence that inflamed tissues are deprived of oxygen, creating an environment where glycolysis is required for survival (Pearce et al. (2013), Immunity 38 (4):633). But so far there is no clinical tool that allows to distinguish fast and feasibly between non-activatable and activatable immune cells.

There is, thus, a need in the art to provide reliable means and methods to test the capability of living cells to adapt to specific environmental conditions; in particular, there is a need to predict the activation propensity of immune cells, preferably in a quick assay not requiring elaborate enrichment and/or activation procedures. In particular, there is a need to provide means and methods avoiding at least in part the drawbacks of the prior art as discussed above.

This problem is solved by methods and devices with the features of the independent claims. Preferred embodiments, which might be realized in an isolated fashion or in any arbitrary combination are listed in the dependent claims.

Thus, the present invention relates to a method of determining a metabolic adaptation of a living entity of interest to a first set of environmental conditions and to a second set of environmental conditions comprising

(a) determining with a first substrate concentration an activity of at least one enzyme comprised in a specimen of said living entity maintained under said first set of environmental conditions and an activity of said at least one enzyme comprised in a specimen of said living entity maintained under said second set of environmental conditions;
(b) determining with a second substrate concentration an activity of at least one enzyme comprised in a specimen of said living entity maintained under said first set of environmental conditions and an activity of said at least one enzyme comprised in a specimen of said living entity maintained under said second set of environmental conditions;
wherein said second substrate concentration is at most twofold, preferably is about equal to or lower than, the K_{M} of said enzyme for said substrate, and wherein at least one of said activities determined in steps (a) and (b) is a non-linear activity; and
(c) determining the metabolic adaptation of said living entity based on comparing at least one non-linear activity determined in step (a) and/or (b) to at least one further activity determined in step (a) and/or (b).

Also, the present invention relates to a method for determining an activation status of immune cells in a test sample comprising said immune cells, comprising

(a) incubating a first subportion of said test sample comprising immune cells under normoxic conditions,
(b) incubating a second subportion of said test sample comprising immune cells under hypoxic conditions,
(c) determining the activities of at least the enzymes high-affinity Pyruvate Kinase (PKHA) and low-affinity Pyruvate Kinase (PKLA) in cells of said first and second subportions,
(d) comparing said activities determined in step (c), and
(c) based on the result of comparison step (d), determining the activation status of the immune cells in said test sample.

The method for determining an activation status, preferably, is an in vitro method. Moreover, it may comprise steps in addition to those explicitly mentioned above. For example, further steps may relate, e.g., to providing a test sample and subportions thereof for steps (a) and (b). Also, secretion of at least one cytokine in the subportion of step (a), of step (b), or in a further subportion may be determined. Moreover, one or more of said steps may be performed by automated equipment. Preferably, the method for determining an activation status is performed as described in EP 2821 790 A1, which is herewith incorporated by reference with respect to its complete disclosure.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “immune cells” is understood by the skilled person to relate to all cells of the normal immune system of a subject, including genetically modified immune cells and non-immune cells with immune cell like properties. Alternative names for immune cells are “white blood cells” and “leukocytes”. Preferably, the term relates to all cells at least potentially involved or related to an immune response of a subject and includes in particular T cells, B cells, NK cells, monocytes, granulocytes, and mixtures thereof. Preferably, the immune cells have the propensity of performing their normal function within the immune system; thus, the immune cells are non-malignant immune cells. More preferably, the immune cells are mononuclear cells, i.e. preferably T cells, B cells, NK cells, monocytes, or mixtures thereof. More preferably, the immune cells are peripheral blood mononuclear cells (PBMCs), i.e. a mixture of in particular T cells, B cells, NK cells, and monocytes isolated from peripheral blood. More preferably, the immune cells are T cells or hematopoietic stem cells, more preferably are CD34+ hematopoietic stem cells. Also preferably, immune cells may be comprised in and/or isolated from other sample types as specified herein below. Genetically modified immune cells are, preferably, immune cells which were genetically modified, preferably genetically modified ex vivo, and which may be used for treatment. Preferred genetically modified immune cells are CAR T-cells and CRISPR-modified immune cells. Non-immune cells with immune cell like properties are known in the art e.g. from Kojima et al. (2017) Nature Chemical Biology, doi:10.1038/nchembio.249

The term “test sample”, as used herein, relates to any composition of matter comprising or suspected to comprise immune cells. Thus, the test sample may be a sample of a body fluid, a sample of separated and/or cultured cells or a sample from a tissue or an organ or a sample of wash/rinse fluid obtained from an outer or inner body surface. Test samples can be obtained by well known techniques and include, preferably, scrapes, swabs or biopsies from any body surface, body cavity, organ or tissue. Such test samples can be obtained by use of brushes, (cotton) swabs, spatula, rinse/wash fluids, punch biopsy devices, puncture of cavities with needles or surgical instrumentation. However, test samples of blood, cerebrospinal fluid, urine, saliva, lacrimal fluid, or stool are also encompassed by the method of the present invention. Tissue or organ test samples may be obtained from any tissue or organ by, e.g., biopsy or other surgical procedures. Immune cells may be enriched from the body fluids or the tissues or organs by separating techniques such as filtration, centrifugation or cell sorting. Preferably, cell, tissue or organ test samples are obtained from those cells, tissues or organs which are known or suspected to comprise immune cells. It is to be understood that the test sample may be further processed in order to carry out the method of the present invention, in particular as specified in the claims, the embodiments, and in the examples. Preferably, the test sample is pre-treated to obtain viable immune cells comprised in said test sample. Preferably, subportions are obtained such that there is a high probability that there is a similar number of immune cells in all subportions obtained, e.g. by providing tissue slices of similar size, preferably obtained from subsequent cuts; or by providing approximately equal amounts of small tissue cuttings, or by enzymatically digesting the test sample (e.g. with trypsin) to isolate cells and providing similar cell numbers. Preferably, the test sample is a blood test sample, preferably of peripheral blood. Also preferably, the test sample is a tissue or bodily fluid sample from an infection site. Also preferably, the test sample is a tumor sample, more preferably a cancer sample, e.g., preferably, a tumor biopsy. Also preferably, the test sample is a sample of lymphatic tissue. It is, however, also envisaged that the test sample is a sample of cultured cells.

Preferably, the test sample comprises a mixture of different types of cells, wherein at least 10%, preferably at least 25%, more preferably at least 50%, even more preferably at least 75%, most preferably at least 85%, of the cells in the test sample are immune cells. Preferably after test sample pre-treatment, at least 25%, more preferably at least 50%, even more preferably at least 75%, most preferably at least 85%, of the cells in the pre-treated test sample and/or in the subportions derived therefrom are immune cells. Preferably, the test sample comprises essentially 100% immune cells, preferably of one type of immune cells, e.g. T cells or hematopoietic stem cells, more preferably CD34+ hematopoietic stem cells, in particular in case the test sample is a sample of enriched and/or cultured immune cells.

The term “activation status”, as used herein, relates to a state of an immune cell of being capable of performing a function in an immune response of a subject, or not. As used herein, an activation status of an immune cell being “active” relates to a state in which said immune cell, optionally after activation or further activation, assumes its characteristic function in an immune response of a subject. Thus, preferably, immune cells in an active activation status are active and optionally further activatable by appropriate stimuli. In the converse, an activation status of an immune cell being “non-active” relates to a state in which said immune cell, even after administration of a cognate antigen, does not assume its characteristic function in an immune response of a subject. Thus, preferably, immune cells in a non-active activation status are anergic. Also preferably, non-active immune cells can be activated by contacting said immune cells with at least one activator compound as specified herein below.

The term “activator compound”, in the context of the present description, is known to the skilled person to relate to compounds providing co-stimulatory signals activating immune cells, in particular anergic immune cells, such as granulocyte-monocyte colony stimulating factor (GM-CSF), preferably human GM-CSF (hGM-CSF), which are, in principle, known in the art. Preferred activator compounds are synthetic and/or recombinant compounds, preferably polypeptides having the biological activity of activating immune cells, preferably activating at least one type of immune cell selected from T cells, B cells, NK cells, monocytes, granulocytes, and mixtures thereof. Preferably, activation of immune cells is measured as increase in proliferation, increase of chemotaxis, and/or as increase of cytokine secretion compared to a control incubated in the absence of the activator compound.

Preferably, the activator compound is a High Mobility Group Box 1 polypeptide or a derivative thereof. As used herein, the term “High Mobility Group Box 1 polypeptide” (HMGB1 polypeptide) relates to a member of the high mobility group of polypeptides known to the skilled person; or to partial sequences or derivatives thereof having the activity of inhibiting the activity of the tetrameric form of PKM2. Preferably, the HMGB1 polypeptide is the human HMGB1 polypeptide (Genbank ACC No: NP_002119.1 GI:4504425, SEQ ID NO:3) or a partial sequence or a derivative thereof having the activity as specified above. Suitable assays for measuring the activities mentioned before are described e.g. in WO 2017/098051, WO 2018/108327, and in the accompanying Examples. The HMGB1 polypeptide may be purified from cells or tissues or it may be chemically synthesized or, preferably, can be recombinantly manufactured and, optionally, biologically or chemically modified. The HMGB1 polypeptide may comprise further amino acids which may serve as a tag for purification or detection, and/or the HMGB1 polypeptide may be comprised by a fusion polypeptide, as specified elsewhere herein. Preferred forms and derivatives of the HMGB1 polypeptide are (i) a polypeptide comprising a HMGB1 polypeptide, preferably at least comprising Box B of HMGB1, more preferably comprising SEQ ID NO:2; (ii) a polypeptide comprising a phosphorylated HMGB1 polypeptide, preferably comprising a polyphosphorylated Box B of human HMGB1; (iii) a polypeptide comprising an oligophosphorylated HMGB1 polypeptide or derivative thereof, wherein at least one of the tyrosine residues corresponding to amino acids Y109, Y144, Y155 and Y162 of the HMGB1 polypeptide was exchanged for a non-phosphorylatable amino acid, preferably glutamine (SEQ ID NO:4), more preferably as described in WO 2017/098051, (iv) a polypeptide comprising a HMGB1 polypeptide, wherein at least two cysteine residues, preferably two cysteine residues of the A-box, more preferably C23 and C45, are covalently connected via an alkyl bridge, preferably an ethyl-bridge; (v) a polypeptide comprising a phospho-mimic HMGB1 polypeptide, wherein at least one of the tyrosine residues corresponding to amino acids Y109, Y144, Y155 and Y162 of the HMGB1 polypeptide was exchanged for an acidic amino acid, preferably glutamate (SEQ ID NO:5), more preferably as described in WO 2018/108327; (vi) a polypeptide comprising an SH-alkylated HMGB1 polypeptide; (vii) any combination and/or mixtures of (i) to (vi). Preferred combinations of (i) to (vi) are combinations of (iv) and (v) and (v) and (vi). Thus, preferably, more preferred derivatives of the HMGB1 polypeptide are (i) a polypeptide comprising a HMGB1 polypeptide, wherein at least two cysteine residues, preferably two cysteine residues of the A-box, more preferably C23 and C45, are covalently connected via an alkyl bridge, preferably an ethyl-bridge; and wherein at least one of the tyrosine residues corresponding to amino acids Y109, Y144, Y155 and Y162 of the HMGB1 polypeptide was exchanged for an acidic amino acid, preferably glutamate; and (ii) a polypeptide comprising an SH-alkylated HMGB1 polypeptide, wherein at least one of the tyrosine residues corresponding to amino acids Y109, Y144, Y 155 and Y162 of the HMGB1 polypeptide was exchanged for an acidic amino acid, preferably glutamate.

Preferably, the term “phosphorylated HMGB1 polypeptide” relates to an HMGB1 polypeptide tyrosine-phosphorylated at at least one, preferably at at least two, more preferably at at least three, most preferably at all four positions selected from Y109, Y144, Y155 and Y162. Also preferably, the polypeptide comprising the B-box motif of the HMGB1 polypeptide is phosphorylated, more preferably tyrosine-phosphorylated at at least one, preferably at at least two, more preferably at at least three, most preferably at all four positions selected from the positions corresponding to Y109, Y144, Y155 and Y162 of the HMGB1 polypeptide. Most preferably, a polyphosphorylated HMGB1 polypeptide is an HMGB1 polypeptide being phosphorylated at the four aforesaid tyrosine residues and additionally being phosphorylated at at least one further residue, preferably serine and/or threonine residue, preferably within the B-Box of the polypeptide.

Preferably, the term “oligophosphorylated HMGB1 polypeptide” relates to an HMGB1 polypeptide wherein at least one of the tyrosine residues corresponding to amino acids Y109, Y144, Y 155 and Y162 of the HMGB1 polypeptide was exchanged for a non-phosphorylatable amino acid, preferably glutamine. Preferably at least one, more preferably at least two, even more preferably at least three, most preferably at all four positions selected from the positions corresponding to Y109, Y144, Y155 and Y162 are exchanged for a non-phosphorylatable amino acid in the oligophosphorylated HMGB1 polypeptide. Most preferably, a oligophosphorylated HMGB1 polypeptide is an HMGB1 polypeptide wherein all four aforesaid tyrosine residues are exchanged for glutamine residues.

Preferably, the term “phospho-mimic HMGB1 polypeptide” relates to an HMGB1 polypeptide wherein at least one of the tyrosine residues corresponding to amino acids Y109, Y144, Y155 and Y162 of the HMGB1 polypeptide was exchanged for an acidic amino acid, preferably glutamate. Preferably at least one, more preferably at least two, even more preferably at least three, most preferably at all four positions selected from the positions corresponding to Y109, Y144, Y155 and Y162 are exchanged for an acidic amino acid in the phospho-mimic HMGB1 polypeptide. Most preferably, a phospho-mimic HMGB1 polypeptide is an HMGB1 polypeptide wherein all four aforesaid tyrosine residues are exchanged for glutamate residues.

The term “redox-fixed HMGB1 derivative” polypeptide, as used herein, relates to an HMGB1 derivative wherein at least one cysteine residue was modified such that formation of a disulfide group is no longer possible. Thus, preferably, in the redox-fixed HMGB1 derivative not necessarily all cysteine residues are modified such that formation of a disulfide group is no longer possible. More preferably, the redox-fixed HMGB1 derivative is a phospho-mimic

HMGB1 derivate. Preferably, the redox-fixed HMGB1 derivative is an SH-alkylated HMGB1 derivative as specified herein below, or, also preferably, the redox-fixed HMGB1 derivative is an HMGB1 derivative wherein at least two cysteine residues are covalently connected via an alkyl bridge, preferably an ethyl-bridge. Preferably, the at least two cysteine residues are two cysteine residues of the A-box of HMGB1, more preferably C23 and C45.

The term “SH-alkylated HMGB1 polypeptide” is understood by the skilled person. Preferably, the term relates to an HMGB1 polypeptide wherein the —SH group of at last one cysteine residue was chemically modified to a non-oxidizable derivative; preferably, said derivative is a iodoacetic acid or a iodoacetamide derivative. Preferably, said derivatization is performed under reducing conditions known to the skilled person. Also preferably, at least two, more preferably at least three, most preferably all accessible cysteine residues are derivatized.

Also preferably, the activator compound is an agent providing HMGB1 polypeptide or a derivative thereof, the term “agent providing HMGB1 polypeptide or a derivative thereof” preferably relating to any agent or composition having the capacity of releasing HMGB1 polypeptide or a derivative thereof as specified herein to a biological system. Preferably, the agent providing HMGB1 polypeptide or a derivative thereof is used at a dose inducing a plasma concentration of from 1 nM to 1000 nM, more preferably of from 10 nM to 250 nM, most preferably of from 25 nM to 150 nM.

Also preferably, the activator compound is an inhibitor of PKM2 activity, more preferably, a compound destabilizing the tetrameric form of PKM2, even more preferably, is P-M2 tide (tyrosine-phosphorylated peptide GGAVDDDYAQFANGG (SEQ ID NO:1)) or a derivative thereof, said derivative of P-M2 tide having the activity of inhibiting PKM2 activity or providing a compound having said activity upon metabolization in the body of a subject. Preferably, the inhibitor of PKM2 activity is used at a concentration of less than 0.1 mM, more preferably less than 0.05 mM, most preferably less than 0.01 mM; or, preferably, at a dose inducing a plasma concentration of less than 0.1 mM, more preferably less than 0.05 mM, most preferably less than 0.01 mM. Also preferably, the modulator of PKM2 activity is used at a concentration of from 0.0005 mM to 0.1 mM, more preferably of from 0.001 mM to 0.05 mM, most preferably of from 0.005 mM to 0.01 mM; or, preferably, at a dose inducing a plasma concentration of from 0.0005 mM to 0.1 mM, more preferably of from 0.001 mM to 0.05 mM, most preferably of from 0.005 mM to 0.01 mM.

Preferably, reference to a polypeptide or a derivative thereof, as used herein, relates to polypeptide itself or a derivative thereof having the activity of being an activator compound and/or being an inhibitor of PKM2; thus, the term, preferably, further includes a polypeptide comprising an amino acid sequence at least 70% identical to the (poly)peptide and having the activity or activities as specified above. Preferably, the term also relates to an agent specifically binding to an immune cell comprising a HMGB1 polypeptide or derivative thereof. Preferably, said agent specifically binding to an immune cell is an antibody, an aptamer, a lectin, or the like. Also preferably, the term agent providing HMGB1 polypeptide or a derivative thereof relates to a HMGB1 secreting cell induced to secrete the HMGB1 polypeptide. Cells which can be induced to secrete HMGB1 and methods for doing so are known in the art; preferred cells which can be induced to secrete HMGB1 are macrophages and NK cells. Also preferably, the term agent providing HMGB1 polypeptide or a derivative thereof relates to an expressible polynucleotide encoding the HMGB1 polypeptide or a derivative thereof. As will be understood by the skilled person, said polynucleotide is, preferably, comprised in a vector or in a host cell.

The term “incubate” is understood by the skilled person and, preferably relates to maintaining cells under conditions permissive for survival and/or proliferation of said cells. Preferred conditions for maintaining immune cells are known to the skilled person and are described herein in the Examples. Preferably, incubation conditions are selected such that the only difference in incubation conditions between the first and the second subportion is the indicated condition, preferably oxygen supply or its surrogate. Preferably, incubation is for of from 6 h to 24 h, more preferably of from 7 h to 15 h, even more preferably of from 10 h to 14 h, most preferably 12 h±1 h. Preferably, the first subportion and the second subportion are incubated for the same time period, i.e. the difference in incubation time between the first subportion and the second subportion, preferably, is at most 1 h, more preferably, is at most 0.5 h, even more preferably, is at most 0.25 h. Preferably, test samples are preconditioned under standard cell culture conditions for at least 12 h, more preferably at least 18 h, most preferably for at least 24 h.

According to the present invention, at least a first subportion of the test sample is incubated under normoxic conditions, i.e. preferably under an atmosphere comprising oxygen at a concentration in a range corresponding to the oxygen content in normal tissue (approx. 1% to 10%) to the oxygen content in air (21% oxygen), preferably under standard conditions. Thus, preferably, the oxygen concentration under normoxic conditions is at least 1%, more preferably at least 5%, more preferably at least 10%; most preferably, the oxygen concentration is 21%. Also preferably, the oxygen concentration under normoxic conditions is of from 1% to 30%, preferably of from 1% to 21%, more preferably of from 5% to 21%, most preferably of from 10% to 21%.

According to the present invention, at least a second subportion of the test sample is incubated under hypoxic conditions, i.e. under an atmosphere comprising an oxygen concentration inducing a hypoxic response in a normal cell. Preferably, the oxygen concentration under hypoxic conditions is at most 0.5%, more preferably at most 0.3%, even more preferably, at most 0.1%, most preferably, is 0%. Preferably, the oxygen concentration under hypoxic conditions is of from 0% to 0.5%, more preferably of from 0% to 0.3%, most preferably of from 0% to 0.1%. Also preferably, hypoxia-like conditions may be induced in the method for determining an activation status by a pharmacological surrogate of hypoxia; pharmacological surrogates of hypoxia in the context of the present invention are pharmacological compounds inhibiting oxidative phosphorylation; pharmacological surrogates of hypoxia, e.g. decouplers, are known in the art. Preferably, the pharmacological surrogate of hypoxia is an inhibitor of pyruvate kinase, preferably of pyruvate kinase M2, more preferably of high-affinity pyruvate kinase M2. Preferably the inhibitor of pyruvate kinase is a peptide comprising, preferably consisting of, a tyrosine-phosphorylated peptide GGAVDDDYAQFANGG (SEQ ID NO:1).

Preferably, normoxic and hypoxic conditions are selected such that a significant difference between said two oxygen concentrations is affecting the at least two test samples. Accordingly, the difference in oxygen concentration between normoxic and hypoxic conditions, preferably, is at least 1%, more preferably, is at least 2%, even more preferably is at least 10%, most preferably is at least 20%.

Methods of determining enzyme activities, in particular of high-affinity Pyruvate Kinase and low-affinity Pyruvate Kinase are known in the art. Preferably, in the method, additional enzyme activities are determined, in particular at least one of Hexokinase, Malate decarboxylase, Lactate dehydrogenase (LDH), and cytochrome c oxidizing Complex IV. Preferably, the activities of at least the enzymes high-affinity Pyruvate Kinase, low-affinity Pyruvate Kinase, and Lactate Dehydrogenase are determined. The enzyme activities can be measured as described in textbooks and known to the skilled person, e.g. from EP 2821 790 A1 Table 1 summarizes preferred assays and reaction conditions for determining relevant enzyme activities.

TABLE 1
Exemplary enzyme assays

| Enzyme activity | | | Reaction |
|---|---|---|---|
| determined | Substrates | Helper Enzyme | measured |
| Pyruvate kinase | 10 mM PEP | Lactate | NADH- |
| low affinity | 1 mM ADP | dehydrogenase | oxidation |
| (PKLA) | 0.5 mM NADH | Stock 6 U/mL | |
| Pyruvate kinase | 0.1 mM PEP | Lactate | NADH- |
| high affinity | 1 mM ADP | dehydrogenase | oxidation |
| (PKHA) | 0.5 mM NADH | 6 U/mL | |
| Lactate | 1 mM pyruvate | | NADH- |
| dehydrogenase | 0.5 mM NADH | | Oxidation |
| (LDH) | | | |

Preferably, the activities determined are determined as relative activities, i.e. as an activity compared to the activity of an enzyme known not to be affected by oxygen availability, e.g. a housekeeping enzyme. More preferably, the activities determined are specific activities, i.e. activity per mass of protein (e.g. expressed as U/mg). It will be understood by the skilled person that the above assay for high-affinity PK does not differentiate between Pyruvate Kinase M1 (product of the PKM1 gene) activity and high-affinity Pyruvate Kinase M2 (product of the PKM2 gene) activity; accordingly, in the assay, the total activity of all Pyruvate kinases present in a subportion of a test sample will be determined, respectively.

As used herein, the term “pyruvate kinase M” or “PKM” relates to one of the products of the PKM gene, preferably the human PKM gene. From the PKM gene, several splice-variants are transcribed, which give rise to isoenzymes. Isoform a, also referred to as Pyruvate kinase M1 (PKM1, Genbank Acc. No: NP_002645.3) is a tetrameric enzyme with high affinity to the substrate phosphoenolpyruvate. A second isoform of pyruvate kinase M is referred to as “pyruvate kinase M2” or “PKM2”. Thus, preferably, the PKM referred to herein is PKM2. Preferably, PKM2 is mammalian PKM2, more preferably human PKM2. Preferably, PKM2 comprises or, more preferably, consists of the amino acid sequence of Genbank Acc NO: AAQ15274.1, preferably encoded by a polynucleotide comprising or consisting of the nucleic acid sequence of Genbank Acc NO: KJ891817.1. PKM2 can exist in a, preferably non-phosphorylated, tetrameric form having a high affinity for its substrate phosphoenolpyruvate; and in a, preferably phosphorylated, dimeric form having a low affinity for its substrate phosphoenolpyruvate. Since conventional activity assays do not discriminate between PKM1 and the high-affinity form of PKM2, the terms “high-affinity pyruvate kinase”, also referred to as “Pyruvate kinase high affinity” or “PKHA” include both of the aforesaid isoenzymes. In contrast, the term “low-affinity pyruvate kinase”, also referred to as “Pyruvate kinase low affinity” or “PKLA” relates to the dimeric form of PKM2.

The step of “comparing” the activities determined according to the present invention is understood by the skilled person. As is understood by the skilled person, activities of the same types of enzymes are compared, respectively. Thus, preferably, an LDH activity under normoxic conditions is compared to an LDH activity under hypoxic conditions, a PKHA activity under normoxic conditions is compared to a PKHA activity under hypoxic conditions, and the like. According to the method of the present invention, a strong change in the activity of either PKHA or PKLA under hypoxic conditions as compared to the activity under normoxic conditions is indicative of immune cells which are active, i.e. which have an activation status being active. Preferably, said change may be an increase or a decrease; also preferably, said change is a change by a factor of at least 1.5, more preferably at least 2, most preferably at least 3. Conversely, a moderate or no change in the activity of either PKHA or PKLA under hypoxic conditions as compared to the activity under normoxic conditions, or a parallel change of both PKHA and PKLA, is indicative of immune cells which are non-active.

Preferably, comparison step (d) comprises calculating ratios of enzymatic activity in the hypoxic subportion to the enzymatic activity in the normoxic subportion. More preferably, aforementioned method step (d) further comprises calculating a ratio of the sum of activities of anaerobic enzyme(s) to the sum of activities of aerobic enzyme(s).

Thus, preferably, step (d) comprises calculation of a metabolic score according to equation (1)

$$MS = \frac{\frac{{A}_{PKLA / hypoxia}}{{A}_{PKLA / normoxia}} + \frac{{A}_{LDH / hypoxia}}{{A}_{LDH / normoxia}}}{\frac{{A}_{PKHA / hypoxia}}{{A}_{PKHA / normoxia}}} , \qquad ( I )$$

with
MS: metabolic score;
A_{PKLA/hypoxia}: activity of low-affinity Pyruvate Kinase in cells under hypoxia;
A_{PKLA/normoxia}: activity of low-affinity Pyruvate Kinase in cells under normoxia;
A_{LDH/hypoxia}: activity of lactate dehydrogenase in cells under hypoxia;
A_{LDH/normoxia}: activity of lactate dehydrogenase in cells under normoxia;
A_{PKHA/hypoxia}: activity of high-affinity Pyruvate Kinase in cells under hypoxia; and
A_{PKHA/normoxia}: activity of high-affinity Pyruvate Kinase in cells under normoxia.

As will be understood, the same calculation can also be provided in a two-step calculation method, wherein, in a first step, preferably, normalized hypoxic/normoxic enzyme activities (A_{X/normalized}), with X=enzyme of interest) are calculated: A_{LDH/normalized}=A_{LDI/hypoxia}/A_{LDH/normoxia}, A_{PKLA/normalized}=A_{PKLA/hypoxia}/A_{PKLA/normoxia}, and A_{PKHA/normalized}=A_{PKHA/hypoxia}/A_{PKHA/normoxia}. In a second step, preferably, the Metabolic Score MS may be calculated according to, e.g., the following equation (11):

$$MS = \frac{{A}_{PKLA / normalized} + {A}_{LDH / n ormalized}}{{A}_{PKHA / normalized}} \qquad ( II )$$

As will be understood from the above, a value of MS corresponding to the number of summands in the dividend, divided by the number of summands in the divisor in the formula applied for calculating metabolic score MS, is indicative of an activation status of immune cells being non-active: in the above example of equation (11), the normalized activities are 1 in case the activity of the respective enzyme is essentially the same under hypoxia and normoxia; i.e. if e.g. A_{LDH/hypoxia}=A_{LDH/normoxia}, then A_{LDH/hypoxia}/A_{LDH/normoxia}=1. Thus, in such case, the value of MS=1+1/1, i.e. MS=2. Thus, in case calculation of the metabolic score MS is performed according to one of equations (I) and (II), an MS of essentially 2 is indicative of a test sample comprising immune cells not actively switching away from oxidative phosphorylation under hypoxia. Thus, such an MS in this case is indicative of immune cells in an activation status being non-active. In the converse, in case calculation of the metabolic score MS is performed according to one of equations (I) and (II), an MS of significantly deviating from 2 is indicative of a test sample comprising immune cells actively switching away from oxidative phosphorylation to glycolysis under hypoxia. Thus, such an MS in this case is indicative of immune cells in an activation status being active. Preferably, in case calculation of the metabolic score MS is performed according to one of equations (I) and (II), a deviation of the MS from a value of 2 by ±0.1, more preferably ±0.25, most preferably ±0.5, is indicative of an activation state being active.

Advantageously, it was found in the work underlying the present invention that by determining the propensity of immune cells to switch from oxidative phosphorylation to glycolysis, the propensity of said immune cells to be or become activated can be determined. Surprisingly, it was found that immune cells switching to glycolysis under hypoxic conditions are active and optionally or further activatable, while immune cells unable to undergo said switch are non-active, mostly anergic. Thus, the method for determining an activation status of immune cells of the present invention allows to predict the ability of immune cells to become activated and to raise an immune response to an antigen.

The definitions made above apply mutatis mutandis to the following. Additional definitions and explanations made further below also apply for all embodiments described in this specification mutatis mutandis.

The present invention further relates to a method of determining a metabolic adaptation of a living entity of interest to a first set of environmental conditions and to a second set of environmental conditions comprising

(a) determining with a first substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₁ and t₂ after starting the determining reaction;
(b) determining with a second substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₃ and t₄ after starting the determining reaction; wherein said second substrate concentration is at most twofold, preferably is about equal to or lower than, the K_{M} of said enzyme for said substrate; and
(c) determining the metabolic adaptation of said living entity based on comparing at least one non-linear activity determined in step (a) and/or (b) to at least one further activity determined in step (a) and/or (b).

The method of determining a metabolic adaptation of the present invention is an in vitro method. The method may comprise steps in addition to those explicitly mentioned above. For example, further steps may relate, e.g., to providing a specimen for steps a) and b), and/or pre-treating said specimen, e.g. by removal of cells, or by enrichment of a group or type of cells, e.g. immune cells. Moreover, the enzyme activities, preferably are determined at at least one, more preferably at least two, still more preferably at least three further substrate concentrations, wherein the substrate concentrations used in said method are, preferably mutually non-identical. Preferably, at least one, more preferably at least two of said further substrate concentrations are in the same range as the second substrate concentration. Thus, preferably, the method comprises at least a further step (b1) determining with a third substrate concentration the activity of said at least one enzyme comprised in said cells maintained under said first environmental condition and determining the activity of said at least one enzyme comprised in said cells maintained under said second environmental condition; wherein, preferably, said third substrate concentration is at most twofold, preferably are about equal to or lower than, the K_{M} of said enzyme for said substrate. More preferably, the second substrate concentration is about equal the K_{M} of said enzyme for said substrate and the third substrate concentration is at most 0.5 fold, preferably at most 0.1 fold, more preferably at most 0.05 fold the K_{M} of said enzyme for said substrate. Moreover, the activity of more than one enzyme may be determined; e.g. as specified herein above, the activity of at least PKLA, PKHA, and LDH may be determined.

Moreover, one or more of said steps may be performed by automated equipment. As will be understood by the skilled person, determining steps (a) and (b) may be performed by automated equipment, e.g. in a high-throughput setting. Moreover, determining step (c) preferably is performed by automated equipment, in particular a computer program, preferably as specified herein below. Thus, preferably, step (c) is computer-implemented, preferably by training an automated machine learning algorithm with the data of steps (a) and (b) of cells having a known metabolic adaptation. Preferably, for determining step (c), a computer-implemented algorithm, preferably an artificial intelligence algorithm, is trained with data of samples with known adaptations to a first environmental condition and to a second environmental condition; preferably, after said training, the computer-implemented algorithm can provide the determining step, preferably without further user-interaction.

As used herein, the term “environmental condition” relates to any measurable parameter exerting influence on a cell. Preferably, the environmental condition is oxygen availability, temperature, pH of the surrounding medium, CO₂ concentration, radiation, presence, absence or concentration of low-molecular weight compounds, osmotic pressure of the surrounding medium, and the like. Preferred low-molecular weight compounds of interest are nutrients and pharmaceutical compounds, in particular chemotherapeutic agents. In accordance, a “set of environmental conditions” is the set of parameters exerting influence on a cell at a given time. Preferably, the first set of environmental conditions and the second set of environmental conditions differ in of from one to five, preferably of from one to four, more preferably of from one to three, even more preferably in one or two, most preferably in exactly one environmental condition, preferably differ only in oxygen availability. It is, however, also envisaged that the first set of environmental conditions and the second set of environmental conditions differ, preferably, in oxygen availability and the presence/absence of a chemotherapeutic compound; or in oxygen availability and presence/absence of an activator compound. Preferably, as specified herein above, the at least one enzyme is pyruvate kinase, preferably pyruvate kinase M2.

The term “metabolic adaptation to an environmental condition” is understood by the skilled person to relate to a measureable change in metabolic composition and/or activity of a cell in response to a change in at least one environmental condition and comprises a measurable change in the activity of at least one enzyme comprised in said cell. Preferably, said adaptation is a change in the activity of at least one enzyme, gene expression, RNA splicing, miRNA expression, epigenetic regulation, or the like. More preferably, said adaptation is a change in the activity of at least one enzyme. Preferably, the environmental condition is oxygen availability; thus, preferably, the metabolic adaptation is a switch from oxidative phosphorylation under normoxia to glycolysis under hypoxia. Thus, the method of determining a metabolic adaptation preferably is used in the method for determining an activation status of immune cells as specified herein above; also preferably, the method of determining a metabolic adaptation is used for determining prognosis of a subject suffering from cancer, in particular leukemia, preferably as described in Gdynia et al. (2018), EBioMedicine 32:125; or for determining whether a subject suffering from inappropriate cellular proliferation is amenable to a treatment with a pharmaceutical compound, in particular a chemotherapeutic agent, preferably as described in Gdynia et al. (2018), EBioMedicine 32:125; or for determining whether a subject suffering from inappropriate cellular proliferation is amenable to a treatment with a modulator of PKM2 activity, preferably as described in WO 2017/09805.

The term “living entity” includes any type of living cell, tissue, organ, or organism, preferably known or suspected to have the ability to adapt to a change in environmental conditions. Preferably, the living entity is an archeal cell, a bacterial cell, or an eukaryotic cell. More preferably, the living entity is a multi-cellular organism, preferably a vertebrate, more preferably a mammal, most preferably a human. In accordance, the term “specimen”, as used herein, relates to a sample of a living entity. In case the living entity is a unicellular or a non-human multicellular, preferably non-mammalian multicellular, organism, the specimen preferably comprises a plurality of said living entities, a living entity or a subportion thereof, or a growth medium comprising or having comprised said living entity. Thus, preferably, the specimen comprises intracellular enzymes of a living entity and/or extracellular enzymes of a living entity. The specimen may also comprise a mixture of living entities. Also preferably, in case the living entity is a multicellular organism, preferably a mammal, more preferably a human, the specimen is a sample of cells or of a bodily liquid of said living entity. Preferably, the specimen comprises cells, more preferably is a blood or tissue sample. Also preferably, the specimen is a cell-free sample, more preferably a plasma or serum sample. Preferably, the specimen is a sample type and is provided as specified herein above in the context of a test sample, which explanations apply to the specimen mutatis mutandis. Preferably, the specimen is a test sample as specified herein above. Preferably, the specimen comprises inappropriately proliferating cells, i.e. cells proliferating to an extent causing a significant deviation from a normal, healthy state of a living entity. Thus, preferably the specimen comprises cancer cells. Also preferably, the specimen comprises immune cells as specified herein above. Preferably, the cells in a specimen are used as they are comprised in the specimen; more preferably, cells of interest are enriched before performing the method of determining a metabolic adaptation, preferably enriched to a concentration as specified herein above for immune cells.

The term “determining the activity” of an enzyme relates to determining progress of the reaction catalyzed by the enzyme over time. Preferred modes of determining reaction progress over time comprise determining increase of at least one of the products over time and/or decrease of at least one of the substrates over time. As will be understood, the term preferably relates to determining an average activity over a time interval. Thus, preferably, determining the activity of an enzyme comprises determining the amount of a product and/or of a substrate at a first time point tₐ and a second time point t_{b} and expressing the activity of the enzyme as the change in concentration of product or of substrate over time interval Δt=t_{b}−tₐ. Preferably, said time interval Δt has a duration of from 0.1 s to 10 min, preferably of from 1 s to 5 min. However, continuous measurement of activity is also envisaged.

According to the method of determining a metabolic adaptation, at least two activities are determined at two non-identical points in time after starting the determining reaction. Thus, preferably, a first activity is determined at time point t₁ (or h) after starting the determining reaction, and a second activity is determined at time point t₂ (or t₄) after starting the determining reaction. The term “starting the determination reaction” is understood by the skilled person as relating to the point in time at which all components required for the determination reaction to occur are combined (to). Preferably, the determination reaction is started by adding the enzyme whose activity is to be determined. Preferably, the interval between the time point of starting the determination reaction and t₁ and/or t₃ is essentially identical, preferably is identical, to the time interval between t₁ and t₂ and/or t₃ and t₄. Preferably, the determinations of steps (a) and (b) are performed in parallel; thus, preferably t₁=t₃ and t₂=t₄. Preferably, at least three activities are determined, more preferably at least four activities are determined, even more preferably at least five activities are determined, most preferably at least six activities are determined at non-identical points in time per combination of enzyme, substrate concentration, and environmental condition. As will be understood, the number of activities to be determined may be selected independently for each combination of enzyme, substrate concentration, and environmental condition; e.g., preferably, in step (a) only two activities may be determined, respectively, and in step (b) at least three activities, more preferably at least four activities, even more preferably at least five activities, most preferably at least six activities may be determined. Preferably, the interval between the time points at which the activities are determined is essentially identical, more preferably is identical. Preferably, the activity at a given point in time is determined by determining the amount of product produced and/or the amount of substrate consumed at a given point in time (tₙ) and subtracting the amount of product produced and/or the amount of substrate consumed at preceding point in time (tₙ₋₁). Preferred measurement durations and time intervals for determining activities can be adjusted by the skilled person in view of the specifications made herein. E.g. in the case of determining PKLA and PKHA according to Table 1 above, activities may be measured over at least 10 min, preferably at least 20 min, more preferably at least 30 min, most preferably over about 30 min, and a preferred measuring interval may be 5 min.

Preferably, at least one enzyme activity is a non-linear activity. As used herein, the term “non-linear activity of an enzyme” relates to an activity of the enzyme at a substrate concentration and/or at a point in time after start of the reaction such that the progress of the reaction over time is non-linear. As will be understood, said non-linear activity preferably is an apparent activity, which is, preferably, lower than the activity which would be determined under standard assay conditions; as will also be understood, the terms “first concentration” and “second concentration” of a substrate relate to the starting concentrations of the substrate in the respective assays, while the substrate concentrations in the assay at the time point of determining enzyme activity may be lower, depending on preceding enzyme activity.

Preferably, the progress of the reaction, preferably increase of product and/or decrease of substrate over time, is non-linear if the amount of product produced and/or the amount of substrate consumed in the assay over a second time interval Δt₂ is at most 80%, preferably at most 70%, more preferably at most 60%, most preferably at most 50% of the amount of product produced in the assay over a first time interval Δt₁, wherein Δt₁=Δt₂ and wherein Δt₁ is before Δt₂. Thus, preferably, an enzyme activity is non-linear if the activity determined is at most 80%, preferably at most 70%, more preferably at most 60%, most preferably at most 50% of a preceding activity, preferably the immediately preceding activity. Thus, preferably, at least one activity, preferably at least two, more preferably at least three, most preferably all four activities of steps (a) and (b) are non-linear activities. Preferably, a plurality of activities of said at least one enzyme comprised in said cells of interest maintained under said first set of environmental conditions is determined over time and/or a multitude of activities of said at least one enzyme comprised in said cells maintained under said second set of environmental conditions is determined with a first starting substrate concentration in step (a), and/or a multitude of activities of said at least one enzyme comprised in said cells of interest maintained under said first set of environmental conditions and/or a multitude of activities of said at least one enzyme comprised in said cells maintained under said second set of environmental conditions is determined over time with a second starting substrate concentration in step (b); wherein at least one of said activities is a non-linear activity. Thus, the method may additionally comprise also determining activity of said enzyme under conditions leading to linear activity. Also preferably, one or both determinations of step (a) and/or of step (b) may be adjusted such that the first activity or activities measured are linear, whereas activities measured later are non-linear.

In determining step (c), at least one non-linear activity determined in step (a) and/or (b) is compared to at least one further activity determined in step (a) and/or (b). Preferably, at least one activity determined in step (b) is non-linear. Thus, preferably, determining the metabolic adaptation of said cells of interest is based on (i) comparing an enzyme activity determined for the first environmental condition in step (a) to an enzyme activity determined for the first environmental condition in step (b); (ii) comparing an enzyme activity determined for the first environmental condition in step (a) to an enzyme activity determined for the second environmental condition in step (b); (iii) comparing an enzyme activity determined for the second environmental condition in step (a) to an enzyme activity determined for the first environmental condition in step (b); (iv) comparing an enzyme activity determined for the second environmental condition in step (a) to an enzyme activity determined for the second environmental condition in step (b); (v) comparing an enzyme activity determined for the first environmental condition in step (b) to an enzyme activity determined for the second environmental condition in step (b), or (vi) any combination of (i) to (v). As will be understood, preferably enzyme activities determined at the same point in time after starting the determining reaction and, more preferably for the same time interval, are compared.

The term “K_{M}” is known to the skilled person as the Michaelis constant of an enzyme for a substrate, which generally indicates the substrate concentration at which the reaction of the enzyme is catalyzed at half-maximal velocity.

Preferably, the first substrate concentration is a substrate concentration conventionally used in the art for determining the activity of the enzyme of interest. Thus, preferably, the first substrate concentration is at least twofold, preferably at least fivefold, more preferably at least tenfold the K_{M} of said enzyme for said substrate. It is, however, also envisaged that the first substrate concentration is in a similar range as, but different from, the second substrate concentration; thus, preferably, the first substrate concentration is at most twofold, preferably is about equal to or lower than, the K_{M} of said enzyme for said substrate and is non-identical to the second substrate concentration. In case the enzyme of interest has two or more substrates or a substrate and a cosubstrate like NAD(P), the above relates to the concentrations of one of said substrates, preferable the non-cosubstrate, while the other substrate concentration(s) are used as conventional in the art. Preferably, in case the at least one enzyme is pyruvate kinase, the substrate is phosphoenolpyruvate (PEP), and the first concentration is 10 mM and the second concentration is 0.1 mM.

Advantageously, and surprisingly, it was found in the work underlying the present invention that by including enzyme activity data from non-linear enzyme reactions, analysis of adaptation of a living entity to changing environmental conditions can be significantly improved.

The invention further discloses and proposes a computer program including computer-executable instructions for performing a method according to the present invention in one or more of the embodiments enclosed herein when the program is executed on a computer or computer network. Specifically, the computer program may be stored on a computer-readable data carrier. Thus, specifically, one, more than one or even all of method steps a) to d) as indicated above may be performed by using a computer or a computer network, preferably by using a computer program.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Further, the invention proposes and discloses a modulated data signal which contains instructions readable by a computer system or computer network, for performing the method according to one or more of the embodiments disclosed herein.

Preferably, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

The present invention also relates to a device comprising a microprocessor and tangibly embedded an algorithm performing at least step (c) of the method of determining a metabolic adaptation when executed on said microprocessor.

A “device” as used herein shall comprise at least the aforementioned means. Moreover, the device, preferably, further comprises means for determining enzyme activities according to steps (a) and/or (b) of the method. Thus, preferably, the device further comprises or is operably connected to a detection unit adapted to perform steps (a) and (b) of the method. The means of the device are, preferably, operatively linked to each other. How to link the means in an operating manner will depend on the type of means included into the device. Preferably, the means are comprised by a single device. The device may accordingly include an analyzing unit for determining the activities and an evaluation unit comprising the microprocessor an the embedded algorithm for processing the resulting data for the assessment. Preferred devices are those which can be applied without the particular knowledge of a specialized clinician, e.g., electronic devices which merely require loading with a sample. Alternatively, the methods for the determination of the at least one biomarker can be implemented into a system comprising several devices which are, preferably, operatively linked to each other. Specifically, the means must be linked in a manner as to allow carrying out the method of the present invention as described in detail above. Therefore, operatively linked, as used herein, preferably, means functionally linked. A preferred system comprises means for determining enzyme activities. Means for determining enzyme activities as used herein encompass means for separating analytes, such as chromatographic devices, and means for analyte determination, such as mass spectrometry devices; also included are means for in-line determination of activity such as photo-optic means, in particular photometric, luminometric, or fluorometric units. Suitable devices are known in the art. Preferably, the device or system further comprises an output unit for outputting the result of the determination and/or of step (c) to a user.

The present invention also relates to a method of activating immune cells, comprising determining an activation status of said immune cells according to the method of the present invention and contacting immune cells in an activation state being non-active with an activator compound.

The method of activating or further activating immune cells, preferably, is an in vitro method. More preferably, the steps relating to determining an activation status of immune cells are in vitro steps, while contacting immune cells in an activation state being non-active with an activator compound may be performed in vitro and/or in vivo. The method, accordingly, may comprise further steps, e.g. providing a test sample for analysis, or administration of additional pharmaceutical compounds, e.g. antibiotics.

Further, the present invention relates to a redox-fixed HMGB1 derivative, to said redox-fixed HMGB1 derivative for use in medicine; and to said redox-fixed HMGB1 derivative for use in activation of immune cells.

The term “redox-fixed HMGB1 derivative” is specified herein above. As specified herein above, the redox-fixed HMGB1 derivative preferably is a phospho-mimic HMGB1 as specified herein above, i.e., preferably, is a redox-fixed phospho-mimic HMGB1 derivative.

In a preferred embodiment, the present invention also relates to a method for determining the activity of at least one enzyme comprising contacting said at least one enzyme to an extract of a fixed cell sample, and determining the activity of said enzyme. In an also preferred embodiment, the present invention further relates to a method of determining a modulation of at least one enzyme activity by an extract of a fixed cell sample, comprising

(i) providing at least a first and a second aliquot of said at least one enzyme;
(ii) contacting said second aliquot with said extract of a fixed cell sample;
(iii) determining the activity of the first aliquot of step (i) and the activity of the second aliquot of step (ii);
(iv) comparing the activities of the first aliquot and the second aliquot determined in step (iii), and thereby
(v) determining a modulation of at least one enzyme activity by an extract of a fixed cell sample.

The aforesaid methods, preferably, are in vitro methods and may comprise further steps, preferably as specified elsewhere herein. Also, one or more steps may be assisted by automated equipment or may be fully automated, preferably as specified elsewhere herein.

The term “cell sample” preferably relates to any sample comprising cells, preferably of immune cells and/or of cancer cells, preferably tumor cells. Preferably, the cell sample is a tissue sample, e.g. of a tumor; also preferably, the cell sample is a test sample as specified herein above.

The term “cancer” is, preferably, understood by the skilled person and relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is tumor recurrence, i.e. relapse. Thus, preferably, the cancer is a solid cancer, i.e. a cancer forming at least one detectable tumor, a metastasis, or a relapse thereof. Preferably, the cancer is selected from the list consisting of aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. Preferably, the cancer is chronic lymphatic leukemia or colon cancer.

The term “fixed” sample, preferably, relates to a sample chemically treated to prevent or slow down deviation of its constituents from the state of the fresh sample. Preferably, the fixation is reversible; thus, preferably, the sample is an aldehyde-fixed sample, more preferably fixed by contacting with formaldehyde and/or glutaraldehyde, more preferably with formaldehyde. Thus, preferably, the fixed cell sample is a sample treated with formalin, i.e. preferably an aqueous solution of formaldehyde, preferably comprising of from 10% (w/w) to 40% (w/w), more preferably about 37% (w/w) formaldehyde. Preferably, the cell sample further is an embedded cell sample, i.e. preferably, a sample of cells embedded into a, preferably waxy, solid, e.g. in order to improve cuttability. Preferably, the cell sample is paraffin-embedded, more preferably is a sample of fixed and paraffin embedded cells.

The term “extract of a fixed cell sample”, preferably, relates to an extract obtained from a fixed cell sample comprising at least polypeptides comprised in said sample. Methods of extracting polypeptides from fixed cell samples are known in the art and related kits including usage information are commercially available. preferably, preparation of an extract from a fixed cell sample comprises heating the sample, preferably at a temperature of from 60° to 90° C., more preferably about 80° C., for of from 10 min to 120 min, preferably about 60 min. As the skilled person will understand, time and temperature required depend on several factors, such of degree of fixation, size of the sample, and the like; the skilled person is able to establish appropriate conditions as required. Preferably, in particular in case the sample is an embedded fixed cell sample, the heating step is preceded by a de-waxing step; appropriate de-waxing conditions are known in the art and include e.g. treatment with a detergent such as sodium dodecylsulfate, and/or organic solvents, e.g. an alcohol.

In a preferred embodiment, the present invention relates to a method of providing a risk classification for a patient suffering from disease, comprising determining the activity of an enzyme according to the present invention and/or determining a modulation of an enzyme activity by an extract of a fixed tissue sample according to the present invention, wherein said fixed cell sample is a sample of said subject.

The term “providing a risk classification”, preferably, relates to assigning a risk to a subject suffering from disease, i.e. an estimation of probability of favorable or unfavorable outcome; preferably, said risk is a risk of progression, treatment failure, relapse, and/or fatal outcome. In accordance, providing a risk classification preferably comprises determining a metabolic adaptation to an environmental condition and/or an activation status of cells, both as specified herein above. Thus, preferably, the method for providing a risk classification comprises the steps of the method of determining a modulation of an enzyme activity by an extract of a fixed tissue sample according to the present invention and determining step (iii) further comprises

(I) determining with a first substrate concentration at least two activities of said first aliquot and at least two activities of said second aliquot, wherein said activities are determined at two non-identical points in time t₁ and t₂ after starting the determining reaction; and
(II) determining with a second substrate concentration at least two activities of first aliquot and at least two activities of said second aliquot, wherein said activities are determined at two non-identical points in time t₃ and t₄ after starting the determining reaction; wherein said second substrate concentration is at most twofold, preferably is about equal to or lower than, the K_{M} of said enzyme for said substrate.

Preferably, the disease is cancer and the fixed cell sample is a sample of fixed blood cells, preferably of fixed immune cells. Also preferably, the disease is cancer and the fixed tissue sample is a cancer sample, preferably a tumor sample.

Preferably, for providing the risk classification, the enzyme activity or activities determined need not necessarily reflect the activity of the enzyme(s) in the cell sample before fixing or their relative regulation. However, it was surprisingly found in the work underlying the present invention that extracts from fixed cell samples of different physiological states (e.g. tumors with different prognosis) can be differentiated by their effect on enzymes added to these extracts. Details are provided herein in Example 8.

In view of the above, the following embodiments are preferred:

### EMBODIMENT 1

A method for determining an activation status of immune cells in a test sample comprising said immune cells, comprising

(a) incubating a first subportion of said test sample comprising immune cells under normoxic conditions,
(b) incubating a second subportion of said test sample comprising immune cells under hypoxic conditions,
(c) determining the activities of at least the enzymes high-affinity Pyruvate Kinase (PKHA) and low-affinity Pyruvate Kinase (PKLA) in cells of said first and second subportions, (d) comparing said activities determined in step (c), and
(c) based on the result of comparison step (d), determining the activation status of the immune cells in said test sample.

### EMBODIMENT 2

The method of embodiment 1, wherein a strong change in the activity of either PKHA or PKLA under hypoxic conditions as compared to the activity under normoxic conditions is indicative of immune cells which are active.

### EMBODIMENT 3

The method of embodiment 1 or 2, wherein a moderate or no change in the activity of either PKHA or PKLA under hypoxic conditions as compared to the activity under normoxic conditions, or a parallel change of both PKHA and PKLA, is indicative of immune cells which are non-active.

### EMBODIMENT 4

The method of any one of embodiments 1 to 3, wherein said immune cells are peripheral blood mononuclear cells (PBMCs), preferably are T-cells or hematopoietic stem cells, more preferably are CD34+ hematopoietic stem cells.

### EMBODIMENT 5

The method of any one of embodiments 1 to 4, wherein said test sample is a blood sample, preferably of peripheral blood, a tumor sample, a sample of lymphatic tissue, or a sample of cultured cells.

### EMBODIMENT 6

The method of any one of embodiments 1 to 5, wherein at least 25%, preferably at least 50%, of the cells in the test sample are immune cells.

### EMBODIMENT 7

The method of any one of embodiments 1 to 6, wherein incubating under normoxic conditions comprises incubating under an atmosphere comprising at least 1% oxygen for at least 12 h±1 h.

### EMBODIMENT 8

The method of any one of embodiments 1 to 7, wherein incubating under hypoxic conditions comprises incubating under an atmosphere comprising at most 0.1% oxygen.

### EMBODIMENT 9

The method of embodiment 8, wherein said inhibitor of pyruvate kinase is a peptide comprising, preferably consisting of, a tyrosine-phosphorylated peptide GGAVDDDYAQFANGG (SEQ ID NO:1).

### EMBODIMENT 10

The method of any one of embodiments 1 to 9, wherein step (c) further comprises determining the activity of lactate dehydrogenase (LDH) in said cells of said first and second subportions.

### EMBODIMENT 11

The method of any one of embodiments 1 to 10, wherein said method further comprises determining secretion of at least one cytokine in the subportion of step (a), of step (b), or in a further subportion.

### EMBODIMENT 12

The method of any one of embodiments 1 to 11, wherein step (d) comprises calculation of a metabolic score according to equation (I)

$$MS = \frac{\frac{{A}_{PKLA / hypoxia}}{{A}_{PKLA / normoxia}} + \frac{{A}_{LDH / hypoxia}}{{A}_{LDH / normoxia}}}{\frac{{A}_{PKHA / hypoxia}}{{A}_{PKHA / normoxia}}} , \qquad ( I )$$

with
MS: metabolic score;
A_{PKLA/hypoxia}: activity of low-affinity Pyruvate Kinase in cells under hypoxia;
A_{PKLA/normoxia}: activity of law-affinity Pyruvate Kinase in cells under normoxia;
A_{LDH/hypoxia}: activity of lactate dehydrogenase in cells under hypoxia;
A_{LDH/normoxia}: activity of lactate dehydrogenase in cells under normoxia;
A_{PKHA/hypoxia}: activity of high-affinity Pyruvate Kinase in cells under hypoxia; and
A_{PKHA/normoxia}: activity of high-affinity Pyruvate Kinase in cells under normoxia.

### EMBODIMENT 13

The method of any one of embodiments 1 to 12, wherein said method further comprises the steps of the method of any one of embodiments 14 to 25.

### EMBODIMENT 14

A method of determining a metabolic adaptation of a living entity of interest to a first set of environmental conditions and to a second set of environmental conditions comprising

(a) determining with a first substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₁ and t₂ after starting the determining reaction;
(b) determining with a second substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₃ and t₄ after starting the determining reaction; wherein said second substrate concentration is at most twofold, preferably is about equal to or lower than, the K_{M} of said enzyme for said substrate; and
(c) determining the metabolic adaptation of said living entity based on comparing at least one non-linear activity determined in step (a) and/or (b) to at least one further activity determined in step (a) and/or (b).

### EMBODIMENT 15

The method of embodiment 14, wherein said first substrate concentration (i) is at least twofold, preferably at least fivefold, more preferably at least tenfold the K_{M} of said enzyme for said substrate; or (ii) wherein said first substrate concentration is at most twofold, preferably is about equal to or lower than, the K_{M} of said enzyme for said substrate and is non-identical to the second substrate concentration.

### EMBODIMENT 16

The method of embodiment 14 or 15, wherein step (c) comprises

(i) comparing an enzyme activity determined for the first environmental condition in step (a) to an enzyme activity determined for the first environmental condition in step (b);
(ii) comparing an enzyme activity determined for the first environmental condition in step (a) to an enzyme activity determined for the second environmental condition in step (b);
(iii) comparing an enzyme activity determined for the second environmental condition in step (a) to an enzyme activity determined for the first environmental condition in step (b);
(iv) comparing an enzyme activity determined for the second environmental condition in step (a) to an enzyme activity determined for the second environmental condition in step (b);
(v) comparing an enzyme activity determined for the first environmental condition in step (b) to an enzyme activity determined for the second environmental condition in step (b), or (vi) any combination of (i) to (v).

### EMBODIMENT 17

The method of any one of embodiments 14 to 16, wherein said method comprises at least a further step (b1) determining the activity of said at least one enzyme comprised in said cells maintained under said first environmental condition and determining the activity of said at least one enzyme comprised in said cells maintained under said second environmental condition with a third substrate concentration; and wherein said third substrate concentration is at most twofold, preferably are about equal to or lower than, the K_{M} of said enzyme for said substrate.

### EMBODIMENT 18

The method of any one of embodiments 14 to 17, wherein said second substrate concentration is about equal the K_{M} of said enzyme for said substrate and wherein said third substrate concentration is at most 0.5 fold, preferably at most 0.1 fold, more preferably at most 0.05 fold the K_{M} of said enzyme for said substrate.

### EMBODIMENT 19

The method of any one of embodiments 14 to 18, wherein at least step (c) is computer-implemented, preferably by training an automated machine learning algorithm with the data of steps (a) and (b) of cells having a known metabolic adaptation.

### EMBODIMENT 20

The method of any one of embodiments 14 to 19, wherein said first environmental condition is normoxia and wherein said second environmental condition is hypoxia and wherein said metabolic adaptation is switch of energy metabolism from oxidative phosphorylation under normoxia to glycolysis under hypoxia.

### EMBODIMENT 21

The method of any one of embodiments 14 to 20, wherein said at least one enzyme is pyruvate kinase, preferably pyruvate kinase M2.

### EMBODIMENT 22

The method of embodiment 21, wherein said substrate is pyruvate and wherein said first substrate concentration is 10 mM and wherein said second substrate concentration is 0.1 mM.

### EMBODIMENT 23

The method of any one of embodiments 14 to 22, wherein steps (a) and (b) comprise determining the activities of at least high-affinity Pyruvate Kinase (PKHA) and low-affinity Pyruvate Kinase (PKLA).

### EMBODIMENT 24

The method of embodiment 23, wherein a strong change in the activity of either PKHA or PKLA under hypoxic conditions as compared to the activity under normoxic conditions is indicative of a successful switch from oxidative phosphorylation under normoxia to glycolysis under hypoxia; and/or wherein a moderate or no change in the activity of either PKHA or PKLA under hypoxic conditions as compared to the activity under normoxic conditions, or a parallel change of both PKHA and PKLA, is indicative of an unsuccessful switch from oxidative phosphorylation under normoxia to glycolysis under hypoxia.

### EMBODIMENT 25

The method of any one of embodiments 14 to 24, wherein at least one of said activities determined in steps (a) and (b) is a non-linear activity.

### EMBODIMENT 26

A device comprising a microprocessor and tangibly embedded an algorithm performing at least step (c) of the method of any one of embodiments 14 to 25 when executed on said microprocessor.

### EMBODIMENT 27

The device of embodiment 26, wherein said device further comprises or is operably connected to a detection unit adapted to perform steps (a) and (b) of the method of any one of embodiments 14 to 25.

### EMBODIMENT 28

A method of activating immune cells, comprising contacting immune cells in an activation state being non-active with an activator compound, wherein said activator compound is selected from a HMGB1 polypeptide or derivative thereof and an inhibitor of PKM2.

### EMBODIMENT 29

The method of embodiment 28, wherein said activator compound is selected from the list consisting of

(i) a polypeptide comprising a HMGB1 polypeptide, preferably at least comprising Box B of HMGB1, more preferably comprising SEQ ID NO:2;
(ii) a polypeptide comprising a phosphorylated HMGB1 polypeptide, preferably comprising a polyphosphorylated Box B of human HMGB1;
(iii) a polypeptide comprising an oligophosphorylated HMGB1 polypeptide or derivative thereof, wherein at least one of the tyrosine residues corresponding to amino acids Y109, Y144, Y155 and Y 162 of the HMGB1 polypeptide was exchanged for a non-phosphorylatable amino acid, preferably glutamine;
(iv) a polypeptide comprising a HMGB1 polypeptide, wherein at least two cysteine residues, preferably two cysteine residues of the A-box, more preferably C23 and C45 are covalently connected via an alkyl bridge, preferably an ethyl-bridge;
(v) a polypeptide comprising a phospho-mimic HMGB1 polypeptide, wherein at least one of the tyrosine residues corresponding to amino acids Y109, Y144, Y155 and Y162 of the HMGB1 polypeptide was exchanged for an acidic amino acid, preferably glutamate;
(vi) a polypeptide comprising an SH-alkylated HMGB1 polypeptide; and
(vii) any combination and/or mixture of (i) to (vi).

### EMBODIMENT 30

The method of embodiment 28 or 29, wherein said activator compound is (i) a polypeptide comprising a HMGB1 polypeptide, wherein at least two cysteine residues, preferably two cysteine residues of the A-box, more preferably C23 and C45, are covalently connected via an alkyl bridge, preferably an ethyl-bridge; and wherein at least one of the tyrosine residues corresponding to amino acids Y109, Y144, Y155 and Y162 of the HMGB1 polypeptide was exchanged for an acidic amino acid, preferably glutamate; and (ii) a polypeptide comprising an SH-alkylated HMGB1 polypeptide, wherein at least one of the tyrosine residues corresponding to amino acids Y109, Y144, Y 155 and Y162 of the HMGB1 polypeptide was exchanged for an acidic amino acid, preferably glutamate.

### EMBODIMENT 31

The method of any one of embodiments 28 to 30, wherein said contacting is preceded by determining an activation status of said immune cells according to the method of any one of embodiments 1 to 13 and wherein immune cells determined to have an activation status being non-active are contacted to said activator compound.

### EMBODIMENT 32

A redox-fixed HMGB1 derivative polypeptide.

### EMBODIMENT 33

The redox-fixed HMGB1 derivative polypeptide of embodiment 32 being a phospho-mimic HMGB1 derivative.

### EMBODIMENT 34

The redox-fixed HMGB1 derivative polypeptide of embodiment 32 or 33, wherein (ii) at least two cysteine residues, preferably two cysteine residues of the A-box, more preferably C23 and C45, are covalently connected via an alkyl bridge, preferably an ethyl-bridge; and wherein at least one of the tyrosine residues corresponding to amino acids Y109, Y144, Y155 and Y162 of the HMGB1 polypeptide was exchanged for an acidic amino acid, preferably glutamate; or (ii) said polypeptide is an SH-alkylated HMGB1 polypeptide, wherein at least one of the tyrosine residues corresponding to amino acids Y109, Y144, Y155 and Y162 of the HMGB1 polypeptide was exchanged for an acidic amino acid, preferably glutamate.

### EMBODIMENT 35

A redox-fixed HMGB1 derivative polypeptide of any one of embodiments 32 to 34 for use in medicine.

### EMBODIMENT 36

A redox-fixed HMGB1 derivative polypeptide of any one of embodiments 32 to 34 for use in activation of immune cells.

### EMBODIMENT 37

A method for determining the activity of at least one enzyme comprising contacting said at least one enzyme to an extract of a fixed cell sample, and determining the activity of said enzyme.

### EMBODIMENT 38

A method of determining a modulation of at least one enzyme activity by an extract of a fixed cell sample, comprising

(i) providing at least a first and a second aliquot of said at least one enzyme;
(ii) contacting said second aliquot with said extract of a fixed cell sample;
(iii) determining the activity of the first aliquot of step (i) and the activity of the second aliquot of step (ii);
(iv) comparing the activities of the first aliquot and the second aliquot determined in step (iii), and thereby
(v) determining a modulation of at least one enzyme activity by an extract of a fixed cell sample.

### EMBODIMENT 39

The method of embodiment 37 or 38, wherein said fixed cell sample is a fixed test sample.

### EMBODIMENT 40

The method of any one of embodiments 37 to 39, wherein said fixed cell sample is an aldehyde-fixed cell sample, preferably a formaldehyde- and/or glutaraldehyde-fixed sample, preferably a formaldehyde-fixed sample.

### EMBODIMENT 41

The method of any one of embodiments 37 to 40, wherein said fixed cell sample is a formalin-fixed cell sample.

### EMBODIMENT 42

The method of any one of embodiments 37 to 41, wherein said fixed cell sample is a formalin-fixed and embedded cell sample, preferably a formalin-fixed and paraffin-embedded cell sample.

### EMBODIMENT 43

The method of any one of embodiments 37 to 42, wherein said fixed cell sample is a fixed tissue sample.

### EMBODIMENT 44

The method of any one of embodiments 37 to 43, wherein said at least one enzyme comprises an enzyme expected or known to have been present in said cell sample before fixation.

### EMBODIMENT 45

The method of any one of embodiments 37 to 44, wherein said at least one enzyme comprises, preferably is, an enzyme of a major metabolic pathway, preferably of glycolysis, citric acid cycle, fatty acid synthesis, nucleotide biosynthesis, or amino acid biosynthesis.

### EMBODIMENT 46

The method of any one of embodiments 37 to 45, wherein said at least one enzyme comprises, preferably is, at least one of high-affinity Pyruvate Kinase (PKHA), low-affinity Pyruvate Kinase (PKLA), and Lactate dehydrogenase (LDH).

### EMBODIMENT 47

A method of providing a risk classification for a patient suffering from disease, comprising determining the activity of an enzyme according to any one of embodiments 37 or 39 to 46 and/or determining a modulation of an enzyme activity by an extract of a fixed tissue sample according to any one of embodiments 38 to 46, wherein said fixed cell sample is a sample of said subject.

### EMBODIMENT 48

The method of embodiment 47, wherein said providing a risk classification comprises determining a metabolic adaptation to an environmental condition and/or an activation status of cells comprised in said fixed cell sample.

### EMBODIMENT 49

The method of embodiment 47 or 48, herein said disease is cancer and wherein said fixed cell sample is a sample of fixed blood cells, preferably of fixed immune cells.

### EMBODIMENT 50

The method of any one of embodiments 47 to 49, wherein said disease is cancer and wherein said fixed tissue sample is a cancer sample, preferably a tumor sample.

### EMBODIMENT 51

The method of any one of embodiments 47 to 50, wherein said method comprises the steps of the method of determining a modulation of an enzyme activity by an extract of a fixed tissue sample according to any one of embodiments 38 to 46, and wherein said determining step (iii) further comprises

(I) determining with a first substrate concentration at least two activities of said first aliquot and at least two activities of said second aliquot, wherein said activities are determined at two non-identical points in time t₁ and t₂ after starting the determining reaction; and
(II) determining with a second substrate concentration at least two activities of first aliquot and at least two activities of said second aliquot, wherein said activities are determined at two non-identical points in time t₃ and t₄ after starting the determining reaction; wherein said second substrate concentration is at most twofold, preferably is about equal to or lower than, the K_{M} of said enzyme for said substrate.

All references cited in this specification are herewith incorporated by reference with respect to their entire disclosure content and the disclosure content specifically mentioned in this specification.

### FIGURE LEGENDS

FIG. 1: Proliferation of PBMCs under the indicated conditions; P-M2 tide: induction of glycolysis by incubation with P-M2 tide; A) control (no activator compound); B) hGM-CSF added; C) to G) HMGB1 or variant thereof added.

FIG. 2: Chemotaxis of PBMCs under hypoxic conditions in the absence and in the presence of P-M2 tide and in the presence of HMGB1 or a variant thereof; Values are % improvement in the presence of a human 4E-HMGB1 with a stable disulfide bond (S-S 4E-hHMGB1) relative to the indicated compound.

FIG. 3: Induction of chemotaxis by HMGB1 cytokines 4E-hHMGB1, 4Q-hHMGB1 and alkylated 4E-hHMGB1 in CD34+ enriched human hematopoetic cord blood cells.

FIG. 4: Schematic representation of patient sample processing in Example 7; Solid tumor (CRC) samples were processed analogously, but fragmented CRC-tissue was incubated instead of a cell suspension in 3 ml RPMI medium. NX=normoxic (aerobic) conditions; AX=anoxic (anaerobic) conditions.

FIG. 5: Pipetting scheme and settings for enzyme kinetic analysis using the EnFin-Test™ kits

FIG. 6: Data evaluation; a matrix that displays time series data (A) was melted (melt function from R) to a single vector where all time points, positive and negative controls (noise) were and both conditions (aerobic/anaerobic) are represented in 168 features. The vectors were used to train machine learning algorithms (B): SVM (top left), bright grey dots represent support vectors at the decision margin, middle gray and dark grey dots the two classes (“0” or “1”), Decision Trees (C5.0 and Random Forest, bottom), showing n-trees used for majority-voting and (feed forward) Neural Networks (top right), here with input and output layer and two hidden layers, dots represent neurons; enzymes added as purified enzymes are referred to as “Avatar enzymes”.

FIG. 7: Activities of the indicated enzymes contacted with the indicated fixed cell extracts in Example 7: A) PKLA/extracts from patients 1 or 2, B) PKLA/extracts from patients 3 or 4, C) PKLA/extracts from patients 5 or 6; D) LDH/extracts from patients 3 or 4, and D) PKHA/extracts from patients 1 or 2.

The following Examples shall merely illustrate the invention. They shall not be construed, whatsoever, to limit the scope of the invention.

### EXAMPLE 1

The cellular model used herein discriminates between an immuno activating environment and an immune suppressive environment. An immuno activating environment comprises supplementation of growth factors/serum and allows growth and survival of blood derived immune cells. An immuno suppressive environment comprises deprivation of growth factors/serum (starvation) and diminishes growth and survival of blood derived immune cells. Growth and survival of immune cells in the patient's blood/tissues are decisive for an appropriately functioning/activatable immune system and are regarded as indexes of immune system function (Pearce et al. (2013), Immunity 38 (4):633; Odegaard et al. (2013), Immunity 38:644). In both conditions a switch to anaerobic glycolysis was induced pharmacologically and proliferation and survival of immune cells was compared to unmodified immune cells. As a proof-of-principle we could show that unmodified immune cells (with no detectable switch to anaerobic glycolysis by means of the herein described blood test) displayed decreased proliferation and survival only in an immune-suppressive environment. Thus by applying the claimed test method to blood derived immune cells one can detect inactivatable/anerg immune cells. Importantly one can discriminate anerg immune cells that turned anerg upon immune-suppressive conditions (e.g. from an immune-suppressed patient) from immune cells that are activatable though exposed to the same immune-suppressive conditions (e.g. from an immune-suppressed patient). This allows specific identification of immune cells that are anergic and, if desirable (e.g. in immuno-paralyzed patients), should be stimulated with appropriate agents.

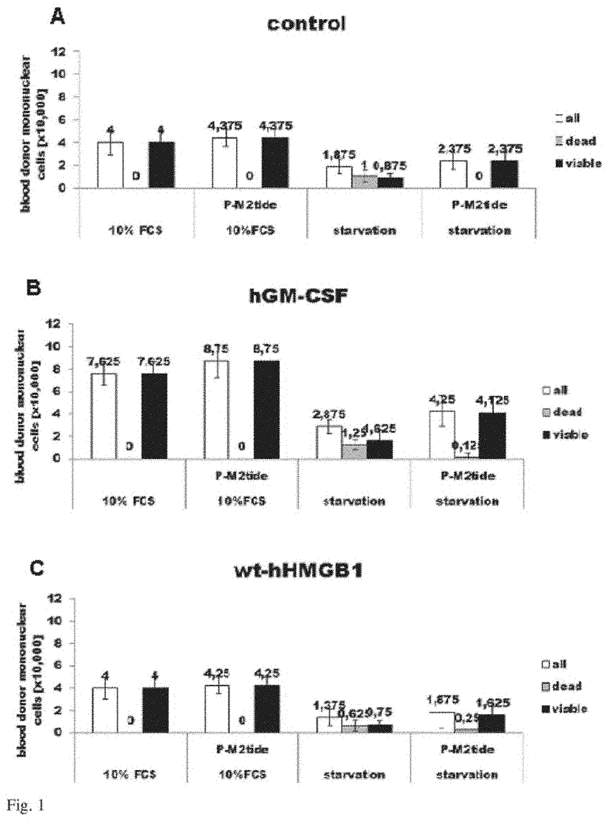

### Experimental Setup (FIG. 1A)

(a) Control 10% FCS: immuno activating environment, immune cells show switch within the central carbon metabolism (CCM) to anaerobic glycolysis, thus patient's immune system is active. Blood test result: 2.67 (significantly changed, reference interval 2.0±0.3).
(b) Control 10% FCS+P-M2 tide: immuno activating environment, cells show switch within CCM to anaerobic glycolysis, thus patient's immune system is active. Blood test result: 0.30 (significantly changed, reference interval 2.0±0.3).
(c) Control starvation: immuno suppressive environment, immune cells show no switch within CCM to anaerobic glycolysis, thus patient's immune system is inactive/anerg. Blood test result: 2.14 (not changed, reference interval 2.0±0.3).
(d) Control starvation+P-M2 tide: immune suppressive environment, immune cells show switch within CCM anaerobic glycolysis, thus patient's immune system is active. Blood test result: 1.37 (significantly changed, reference interval 2.0±0.3).

The switch from OXPHOS to glycolysis occurs upon activation of immune cells (Palsson-McDermott et al. (2015), Cell Metabolism 21:65; Pearce et al. (2013), Immunity 38 (4):633). To measure the maximum possible switch one has to eliminate OXPHOS completely, this is achieved herein by cultivating the immune cells under oxygen deficiency (anoxia), where OXPHOS is inactive due to lack of oxygen as substrate.

### EXAMPLE 2

To evaluate to what extent and by which agent anerg immune cells could be stimulated we used recombinant human GM-CSF (20 ng/ml, FIG. 1 B) or recombinant/synthetic variants of the immune system stimulating human HMGB1 cytokine (200 nM, FIG. 1 C-G).

Mononuclear human blood donor cells with an increased test score (i.e. showing a switch within CCM from OXPHOS to glycolysis measured by the blood test of this invention) did not die under immuno-suppressive conditions (starvation) in control cells. Treatment with human granulocyte monocyte colony stimulating factor (GM-CSF) partly rescued immuno-suppressed cells with no switch to anaerobic glycolysis (unchanged test score) whereas immune-suppressed cells displaying a switch to anaerobic glycolysis were fully rescued from cell death and showed increased proliferation (and survival) (compared to unstimulated control samples). Wildtype human recombinant HMGB1 (high mobility group box 1 protein), a potent immuno-stimulating cytokine and DAMP failed to increase proliferation and survival of immune cells. Recombinant human 4E-HMGB1 (a variant of HMGB1 with four tyrosine residues exchanged for glutamate residues as specified herein above; WO 2018/108327) increased proliferation of non suppressed immune cells showing a switch to anaerobic glycolysis and also increased survival and proliferation of immuno-suppressed immune cells (starved) showing a switch to anaerobic glycolysis. However, compared with hGM-CSF stimulated cells, it had moderate effects on proliferation and survival of suppressed immune cells showing this switch. On the contrary recombinant human 4Q-HMGB1 (a variant of HMGB1 with four tyrosine residues exchanged for glutamine residues as specified herein above; WO2017/098051) showed a weak stimulation of immune-suppressed cells with no switch to anaerobic glycolysis compared to 4E-hHMGB1 and no significant increase in stimulation of immune-suppressed cells that had a switch to anaerobic glycolysis (compared to control cells).

To potently increase stimulation of the immuno-suppressed cells (to at least a level like in activatable immune cells that were exposed to immune-suppressive conditions) we changed the chemical structure of the most promising immune-stimulating recombinant HMGB1 variant, the 4E-hHMGB1, in two ways: (i) by changing the SH (sulfhydryl)-residues (residues Cys-23, Cys-45 and Cys-106 now being irreversibly reduced via alkylation in 4E-hHMGB1) to stable alkylated residues (non-oxidizable (reduced-alkylated) form; alkyl 4E-hHMGB1) and (ii) by introducing permanent disulfide bonds into the protein structure (A-Box domain, residues Cys-23 and Cys-45 now forming a stable disulfide bond in 4E-hHMGB1; S-S 4E-hHMGB1). The alkyl 4E-hHMGB1 stimulation resulted in increased survival and proliferation of immuno-suppressed immune cells (compared to control, hGM-CSF and wt-hHMGB1), however, the effect was minor than with (non alkylated) 4E-hHMGB1. The stimulation with the synthetic recombinant S-S 4E-hHMGB1 variant showed best survival and proliferation effects on both activated (with a switch to anaerobic glycolysis) and not activated (showing no switch to anaerobic glycolysis) immuno-suppressed immune cells. It activated proliferation in non suppressed immune cells to a lesser extent than the 4E-hHMGB1 counterpart, however, the challenge of this invention was (a) to identify immune cells that were inactive/anerg (no switch to anaerobic glycolysis) when exposed to immune-suppressive conditions and (b) to show they could be potently activated by new compounds presented in this invention thereby cancelling the immune-suppressive effect.

### EXAMPLE 3

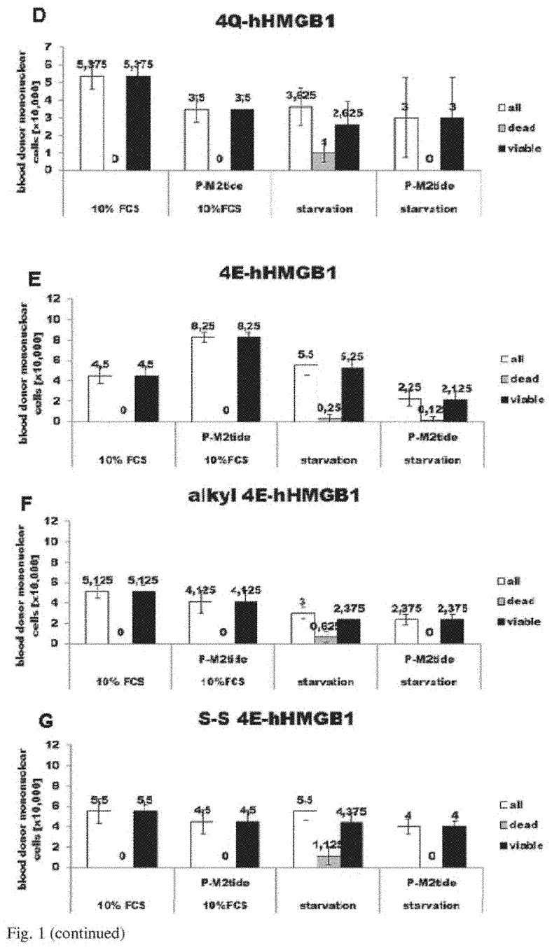

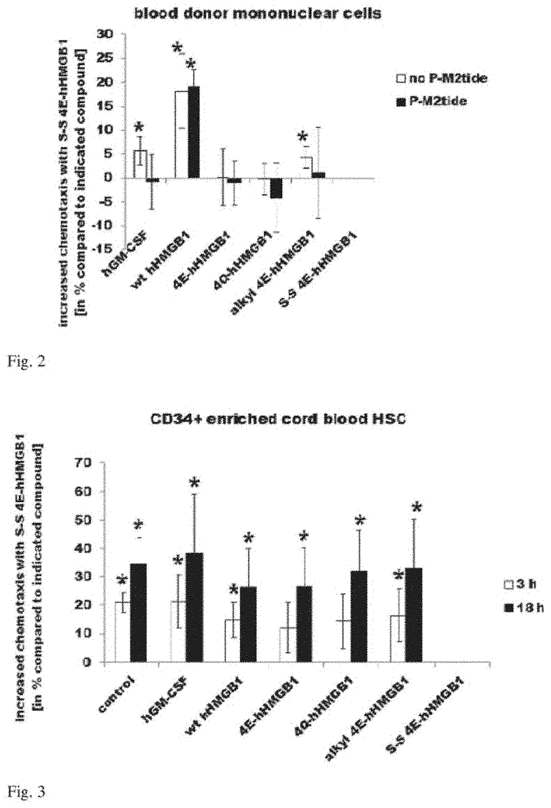

In further experiments using the synthetic and/or recombinant HMGB1 variants we examined also chemotaxis on differentiated immune cells (mononuclear blood donor cells, FIG. 2). S-S 4E-hHMGB1 increased chemotaxis in serum starved (immuno suppressed) immune cells that were inactive/anerg, i.e. they displayed no switch to anaerobic glycolysis (as defined by the test score of this invention) compared to hGM-CSF, wt hHMGB1 cytokine and alkylated 4E-hHMGB1. Moreover, synthetic S-S 4E-hHMGB1 had superior chemotactic effects (compared to the wildtype hHMGB1 cytokine) on blood donor mononuclear cells that were exposed to immuno-suppressive conditions but active (that showed a switch towards anaerobic glycolysis). Although S-S 4E-hHMGB1 failed to induce more chemotaxis than possible with the three recombinant/synthetic HMGB1 cytokines 4E-hHMGB1 4Q-hHMGB1 and alkylated 4E-hHMGB1 it was superior to all indicated compounds in inducing chemotaxis in CD34+ enriched human hematopoetic cord blood cells (HSC, FIG. 3). Chemotactic stimulation of (hematopoetic) stem cells is an important feature of immune system stimulation yin response to pathogen-induced inflammation, autoimmune disease, sepsis, prevention of development, progression or recurrence of cancer, immunodeficiency disorders, prevention of exhaustion of immune cells, tissue repair (proliferation and/or migration of immune stem cells within/to the wounded/ischemic tissue (e.g. blood, bone marrow, heart, coronary arteries, vessels, bones, brain, nerves, myelin sheaths) (Palsson-McDermott et al. (2015), Cell Metabolism 21:65).

In summary we showed that anerg blood donor immune cells cultivated under immune-suppressive conditions (Model Summary (c)) could be potently stimulated by treatment with S-S 4E-hHMGB1, best-in-class compared to other indicated compounds, also regarding increased chemotaxis of human mononuclear immune cells and human CD34+ enriched cord blood hematopoetic stem cells. We also provide proof-of-principle ex vivo data on human immune cells showing that (i) modulation of PKM2 activity by pharmacological inhibition of PK la by specifically blocking the PK tetramer (using a well-characterized phosphotyrosine peptide called P-M2 tide; GGAVDDDpYAQFANGG)) enables immune cells to shift from OXPHOS to anaerobic glycolysis and (ii) that the occurrence of this shift enables immune cells to proliferate/survive/migrate under immuno-suppressive conditions.

One important feature of this blood test is that it shows the individual's unique immune cell activation status. That is achieved by assessing the change of enzyme activities within the individual's samples under two conditions (normoxic and anoxic), thus not using absolute values (that would require reference values of other individuals to perform inter-individual comparison of immune status) but relative values (ratios).

### EXAMPLE 4: EXPERIMENTAL DETAILS

### EXPERIMENT 1

Healthy blood donor mononuclear lymphocytes were cultured under normoxia (21%) and anoxia (0%). Enzyme activities of PK low affinity (PK la), PK high affinity (PK ha) and Lactate dehydrogenase (LDH) were measured in the homogenates. Under anoxic cell culture conditions macromolecule (amino acids, fatty acids, RNA/DNA) and energy (ATP) synthesis has to be fueled by glucose intermediates from central carbon metabolism (CCM). A significant change in enzyme activities from normoxia to anoxia was indicative of an increased shift to anaerobic glycolysis shown by increased proliferation (cell culture supplemented with growth factors) and survival (cell culture not supplemented with growth factors, i.e. serum starvation conditions) of immune cells. One important finding was that when immune cells that displayed no shift to anaerobic glycolysis were suppressed by starvation (no supplement of growth factors), these consequently died and/or did not proliferate (compared to the unsuppressed control and the suppressed control showing a shift to anaerobic glycolysis). Noncleavable cross-linking of sulfhydryl groups was done with 3×800 μl 4E-hHMGB1 (410 μg/ml in PBS, pH 7.4) using the midi-dialyse system, adding 8 μl 0.5 M DTT (1 h, 22° C.) followed by dialysis with 500 ml ice-cold PBS+5 mM EDTA (pH 7, 1 h, 4° C.) and dialysis with 500 ml ice-cold PBS+5 mM EDTA (pH 7, overnight, 4° C.). Then 8 μl of BMOE (bis(maleimido)ethane, Thermo Fisher) was added (1 h, 22° C.) followed by 8 μl 0.5 DTT. The solution was dialyzed for 1 h with 500 ml ice-cold PBS+5 mM EDTA (pH 7, 4° C.) and overnight with 500 ml ice-cold PBS+5 mM EDTA (pH 7, 4° C.). Alkylation of sulfhydryl groups was done with 3×800 μl 4E-hHMGB1 (410 μg/ml in PBS, pH 7.4) using the midi-dialyse system, adding 8 μl 0.5 M DTT (0.1 h, 22° C.) followed by dialysis with 500 ml ice-cold PBS+5 mM EDTA (pH 8, 1 h, 4° C.) and dialysis with 500 ml ice-cold PBS+5 mM EDTA (pH 8, overnight, 4° C.). Then 8 μl of 0.5 M iodaceteamide (30 mM, 22° C., without light) was added followed by 8 μl 0.5 M DTT. The solution was dialyzed for 1 h with 500 ml ice-cold PBS+5 mM EDTA (pH 7.4, 4° C.) and overnight with 500 ml ice-cold PBS+5 mM EDTA (pH 7.4, 4° C.). P-M2 tide oligopeptide (Gly-Gly-Ala-Val-Asp-Asp-Asp-pTyr-Ala-Gln-Phe-Ala-Asn-Gly-Gly) was purchased from Enzo Life Sciences. (n=8).

### EXPERIMENT 2

Chemotactic capacity of different human HMGB1 forms (expressed in HEK cells as described before and, in case of synthetic alkyl 4E-hHMGB1 and synthetic S-S 4E-hHMGB1 modified chemically on their Cysteine-residues) or human recombinant GM-CSF towards healthy blood donor mononuclear lymphocytes (isolated by the recommended Ficoll gradient standard procedure using Ficoll-Paque™) with or without P-M2 tide (from Enzo Life Sciences) oligopeptide (Gly-Gly-Ala-Val-Asp-Asp-Asp-pTyr-Ala-Gln-Phe-Ala-Asn-Gly-Gly, SEQ ID NO:1). The distinct HMGB1 variants display different binding properties to the allosteric center of PK la enzyme. The assay was performed according to the instructions of the manufacturer using Corning Plate HTS Transwell (CLS3374-2 EA) systems with 5 μm pores (10,000 cells per well, 3 h, n=2).

### EXPERIMENT 3

Chemotaxis of human HMGB1 variants towards fresh human CD34+ enriched cord blood hematopoetic stem cells (HSC). HSCs were isolated according to (Wein et al. (2010), Stem Cell Res 4:129) and immediately used for the chemotaxis assay. Briefly, mononuclear cells (MNC) were isolated by density gradient centrifugation with the Ficoll-hypaque technique (Biochrom, Berlin, Germany). CD34+ cells were purified by positive selection with a monoclonal anti-CD34 antibody using magnetic microbeads on an affinity column with the AutoMACS system (all Miltenyi Biotec, Bergisch-Gladbach, Germany). Reanalysis of the isolated cells by flow cytometry revealed a purity of >95% CD34+ cells. The assay was performed according to the instructions of the manufacturer using Corning Plate HTS Transwell (CLS3374-2 EA) systems with 5 μm pores (10,000 cells per well, 311 and 18 h, n=3).

### EXAMPLE 5

Code example for correct classification of leukemia patients (n=22; classification problem=responders vs non-responders to chemotherapy plus rituximab treatment within two years after beginning of the treatment) and colorectal cancer patients (n=101; classification problem=DFS after curative surgery (resection of the primary tumor plus metastasis if present) within two years after surgery); 100% accuracy, p<0.05:

##########
# Caret
# http://dataaspirant.com/2017/01/19/support-vector-machine-classifier-implementation-r-caret-package/
##########
library(gtools)
library(openxlsx)
Iibrary(reshape2)
library(caret)
library(e1071)
library(plyr)
library(DMwR)
library(randomForest)
baseDirectory <− “resources”
myWD <− “D:\\Daten\\Sicherheitskopie_011117\\machine learning\\machineLearning”
setwd(myWD)
source(file.path(myWD, “EnfinMachineLearningHelperLibrary.R”))
################################################################################
################################################################################
################################################################################
# CLL
################################################################################
################################################################################
################################################################################
#LBP_CLL_BaseDirectory <− file.path(baseDirectory, “LBP_CLL”)
LBP_CLL_BaseDirectory <− file.path(baseDirectory, “LBP_CLL_EDITED”)
cll_filepaths <− list.files(LBP_CLL_BaseDirectory,

recursive = TRUE,
full.names = TRUE,
include.dirs = TRUE,
pattern = “*.xlsx”)

cll_filepaths <− file.path(myWD, cll_filepaths)
myRawData <− list( )
for (i in 1:length(cll_filepaths)) {

sheetNames <− getSheetNames(cll_filepaths[i])
for (j in 1:length(sheetNames)) {

sheetNamesFixed <− gsub(“ ”, “”, sheetNames[j])
myRawData[[sheetNamesFixed]] <− read.xlsx(cll_filepaths[i], sheetNames[j], rowNames = TRUE)

}

}
dataPerPatientCLL <− MeltDataPerPatient(myRawData)
################################################################################
# Importing and adding classes
################################################################################
dataPerPatientCLLforClassification <− as.data.frame(t(dataPerPatientCLL))
# Preparing the aditional data from classification and regression
classesForCLL <− read.xlsx(file.path(baseDirectory, “templateDataForCLL_gg.xlsx”))
classesForCLL[classesForCLL == “na”] <− NA
classesForCLL$start <− convertToDate(classesForCLL$start)
classesForCLL$end <− convertToDate(dassesForCLL$end)
classesForCLL$TFSdays <− classesForCLL$end - classesForCLL$start
dataPerPatientCLLforClassification <− NA
dataPerPatientCLLforClassification <− rbind(dataPerPatientCLL, classesForCLL$class)
dataPerPatientCLLforClassification <− dataPerPatientCLLforClassification[,
lis.na(dataPerPatientCLLforClassification[nrow(dataPerPatientCLLforClassification),])]
dataPerPatientCLLforClassification <− t(dataPerPatientCLLforClassification)
dass(dataPerPatientCLLforClassification) <− “numeric”
colnames(dataPerPatientCLLforClassification) <−c(paste0(“f”, 1:168), “class”)
dataPerPatientCLLforClassification <− as.data.frame(dataPerPatientCLLforClassification)
dataPerPatientCLLforClassification$class <− factor(dataPerPatientCLLforClassification$class)
data <− dataPerPatientCLLforClassification
newData <− SMOTE(class ~ ., data)
mySeed <− 3233
#mySeed <− 1
set.seed(mySeed)
proportion <− 0.7
intrain <− createDataPartition(y = newData$class, p = proportion, list = FALSE)
training <− newData[intrain,]
testing <− newData[-intrain,]
training[[“class”]] = factor(training[[“class”]])
trctrl_repeatedcv <− trainControl(

method = “repeatedcv”,
number = 10.
repeats = 3)

trctrl <− trctrl_repeatedcv
grid <− expand.grid(C = c(0.01, 0.05, 0.1, 0.25, 0.5, 0.75, 1, 1.25, 1.5, 1.75, 2,5))
svm_Linear_Grid <− train(class ~., data = training,

method = “svmLinear”,
trControl = trctrl,
preProcess = c(“center”, “scale”),
tuneGrid = grid,
tuneLength = 10)

svm_svmRadial <− train(class data ~., = training,

| method = “svmRadial”, # Radial kernel | |
|---|---|
| # | tuneLength = 9, # 9 values of the cost function |

```
  preProc = c(“center”,“scale”), # Center and scale data
  trControl=trctrl)
```

rf_random <− train(class ~,. data = training,

method = “rf”, # Random forest
preProc = c(“center”,”scale”), # Center and scale data
trControl = trctrl)

test_pred_svm_radial <− predict(svm.svmRadial, newdata = testing)
test_pred_ svm_linear <− predict(svm_Linear_Grid, newdata = testing)
test_pred_rf <− predict(rf_random, newdata = testing)
confusionMatrix(test_pred_svm_radial, testing$class, positive = “1”)
confusionMatrix(test_pred_svm_linear, testing$class, positive = “1”)
confusionMatrix(test_pred_rf, testing$class, positive = “1”)
################################################################################
################################################################################
################################################################################
# CRC
################################################################################
################################################################################
################################################################################
impact_CRC_baseDirectory <− file.path(baseDirectory, “IMPACT_EnFin”)
crc_filepaths <− list.files(impact_CRC_baseDirectory,

recursive = TRUE,
full.names = TRUE,
include.dirs = TRUE,
pattern = “*.xlsx”)

crc_filepaths <− file.path(myWD, crc_filepaths)
crc_filepaths <− crc _filepaths[!grepl(“CRC BA IMP”, crc_filepaths)]
dataPerPatientCRC <− NULL
dataPerPatientCRCColumnNames <− NULL
listOfPlattesIdentifiers <− paste0(“Platte ”, 1:(length(crc_filepaths)), “,”)
for (platteIdentifier in listOfPlattesIdentifiers) {

kitFilepath <− grepl(platteIdentifier, crc_filepaths)
rawData <− read.xlsx(crc_filepaths[kitFilepath], rowNames = TRUE)
listOfPatients <− SortData(rawData)
for(patient in names(listOfPatients)) {

patientIdentifier <− paste(platteIdentifier, patient)
meltedPatient <− melt(listOfPatients[patient])
dataPerPatientCRC <− cbind(dataPerPatientCRC, meltedPatient[, 2])
dataPerPatientCRCColumnNames <− c(dataPerPatientCRCColumnNames, patientIdentifier)

}

}
colnames(dataPerPatientCRC) <− dataPerPatientCRCColumnNames
colnames(dataPerPatientCRC) <− gsub(“,”, “_”, gsub(“ ”, “”, colnames(dataPerPatientCRC), fixed =
TRUE), fixed = TRUE)
colnames(dataPerPatientCRC) <− gsub(“Platte”, “Kit”, colnames(dataPerPatientCRC), fixed = TRUE)
daysPerYear <− 365
threshold <− 2 * daysPerYear
#dataPerPatientCRCforRegressionTMP <− dataPerPatientCRC
classesForCRC <− read.xlsx(file.path(baseDirectory, “templateDataForCRC_gg.xlsx”))
dataPerPatientCRCforClassification <− as.data.frame(t(dataPerPatientCRC))
# classification is representing recurrenceWithinTwoYears
#dataPerPatientCRCforClassification <− cbind(dataPerPatientCRCforClassification, class =
as.integer(classesForCRC$DFS < threshold))
dataPerPatientCRCforClassification <− cbind(dataPerPatientCRCforClassification, class =
as.integer(classesForCRC$DFS < threshold))
dataPerPatientCRCforClassification$class <− factor(dataPerPatientCRCforClassification$class)
data <− dataPerPatientCRCforClassification
newData <− SMOTE(class ~ ., data)
mySeed <− 3233
#mySeed <− 1
set.seed(mySeed)
proportion <− 0.7
intrain <− createDataPartition(y = newData$class, p = proportion, list = FALSE)
training <− newData[intrain,]
testing <− newData[-intrain,]
training[[“class”]] = factor(training[[“class”]])
trctrl_repeatedcv <− trainControl(

method = “repeatedcv”,
number = 10,
repeats = 3)

trctrl <− trctrl_repeatedcv
grid <− expand.grid(C = c(0.01, 0.05, 0.1, 0.25, 0.5, 0.75, 1, 1.25, 1.5, 1.75, 2,5))
svm_Linear <− train(class ~., data = training,

method = “svmLinear”,
trControl = trctrl,
preProcess = c(“center”, “scale”),
tuneGrid = grid,
tuneLength = 10)

svm_svm_radial <− train(class ~., data = training,

| method = “svmRadial”, # Radial kernel | |
|---|---|
| # | tuneLength = 9, # 9 values of the cost function |

preProc = c(“center”,“scale”), # Center and scale data
trControl=trctrl)

rf_random <− train(class ~., data = training,

method = “rf”, # Random forest
preProc = c(“center”,“scale”), # Center and scale data
trControl = trctrl)

test_pred_svm_radial <− predict(svm_svm_radial, newdata = testing)
test_pred_svm_linear <− predict(svm-Linear, newdata = testing)
test_pred_rf <− predict(rf_random, newdata = testing)
confusionMatrix(test_pred_svm_radial, testing$class, positive = “1”)
confusionMatrix(test_pred_svm_linear, testing$class, positive = “1”)
confusionMatrix(test_pred_rf, testing$class, positive = “1”)
####################################################
# Helper functions
####################################################
slice <− function(input, by=2) {

starts <− seq(1,length(input),by)
tt <− lapply(starts, function(y) input[y:(y+(by-1))])
llply(tt, function(x) x[!is.na(x)])

}
SortData <− function(rawData) {
#baseDirectory <− “/Users/tanovsky/wip/Enfin/KitAnalyzer/”
#inputDataFilepath <− file.path(baseDirectory,“input”)
#outputDataFilepath <− file.path(baseDirectory, “output”)
#setwd(baseDiredory)
#rawDataFilepath <− file.path(inputDataFilepath, “Beipiel-Rohdaten.xlsx”)
#test_that(“Input file is accessible”, {
# expect_equal(file.exists(rawDataFilepath), TRUE)
#})
#rawData <− read.xlsx(rawDataFilepath, colNames = TRUE, rowNames = TRUE)
# For test
# rawData <−myData( )
wellColumns <− “”“”
kMaxNumberOfPatients <− 4
kColumnsPerPatient <− 3
timeIntervals <− seq(from = 0, to = 30, by = 5)
timeIntervalLabels <− colnames(rawData)
# generate a vector with all the wells that should be splitted by correspondent patient
orderedWells <− c( )
for (patient in 1:kMaxNumberOfPatients) {

endColumn <− patient * kColumnsPerPatient
beginColumn <− endColumn - kColumnsPerPatient + 1
for(patientColumn in c(beginColumn: endColumn)) {

for (wellRow in LETTERS[1:8]){

orderedWells <− c(orderedWells, paste0(wellRow, patientcolumn))

}

}

}
####################################################
# Generate tables for Patients and fill with time values
####################################################
patientTables <− list( )
patientIdentifier <− paste0(“Patient”, 1:kMaxNumberOfPatients)
#splittedWellsPerPatient <− split(orderedWells, kMaxNumberOfPatients * )
splittedWellsPerPatient <− slice(orderedWells, 24)
for (i in 1:kMaxNumberOfPatients) {

patientTables[[patientIdentifier[i]]] = data.frame(matrix(ncol = length(timeIntervalLabels),

nrow = length(orderedWells)/kMaxNumberOfPatients))

rownames(patientTables[[patientIdentifier[i]]]) <− splittedWellsPerPatient[[i]]
colnames(patientTables[[patientIdentifier[i]]]) <− timeIntervalLabels

# patientTables[[patientIdentifier[i]]] <− cbind(patientTables[[patientIdentifier[i]]], Well =
rownames(patientTables[[patientIdentifier[i]]]))

for (wellIdentifer in rownames(patientTables[[patientIdentifier[i]]])) {

for (timeIdentifier in timeIntervalLabels) {

patientTables[[patientIdentifier[i]]][wellIdentifer, timeIdentifier] <− rawData[wellIdentifer, timeIdentifier]

}

}

}
# Clean the empty patients..(the whole matrix has ‘NA's)
for (i in names(patientTables)) {

if (all(is.na(patientTables[[i]]))) {

patientTables[i] <− NULL

}

}
return(patientTables)
}

### EXAMPLE 6

Examples of training set results for the leukemia classification problem of Example 5 (classification of responders vs non-responders to chemotherapy plus rituximab treatment within two years after beginning of the treatment) using random forest, decision trees (C5.0) and SVM (support vector machine) with radial and linear kernels. Receiver operating curves values reach 100% or near 100%:

>fit.rf

### Random Forest

49 samples
168 predictors
2 classes: ‘no’, ‘yes’
Resampling: Cross-Validated (10 fold, repeated 10 times)
Summary of sample sizes: 44, 44, 44, 44, 44, 44, . . .
Resampling results across tuning parameters:
mtry ROC Sens Spec
2 1 0.990 1
85 1 1.000 1
168 1 0.995 1
ROC was used to select the optimal model using the largest value.
The final value used for the model was mtry=2.
>fit.c50

### C5.0

49 samples
168 predictors
2 classes: ‘no’, ‘yes’
Resampling: Cross-Validated (10 fold, repeated 10 times)
Summary of sample sizes: 44, 44, 44, 44, 44, 45, . . .
Resampling results across tuning parameters:

| model | winnow | trials | ROC | Sens | Spec |
|---|---|---|---|---|---|
| rules | FALSE | 1 | 0.9658333 | 1.0000000 | 0.9316667 |
| rules | FALSE | 10 | 0.9658333 | 1.0000000 | 0.9316667 |
| rules | FALSE | 20 | 0.9658333 | 1.0000000 | 0.9316667 |
| rules | TRUE | 1 | 0.8808333 | 0.9116667 | 0.8483333 |
| rules | TRUE | 10 | 0.9150000 | 0.9466667 | 0.8650000 |
| rules | TRUE | 20 | 0.9141667 | 0.9466667 | 0.8650000 |
| tree | FALSE | 1 | 0.9937500 | 1.0000000 | 0.9316667 |
| tree | FALSE | 10 | 0.9937500 | 1.0000000 | 0.9316667 |
| tree | FALSE | 20 | 0.9937500 | 1.0000000 | 0.9316667 |
| tree | TRUE | 1 | 0.9172222 | 0.9116667 | 0.8550000 |
| tree | TRUE | 10 | 0.9330556 | 0.9466667 | 0.8616667 |
| tree | TRUE | 20 | 0.9322222 | 0.9466667 | 0.8650000 |

ROC was used to select the optimal model using the largest value.
The final values used for the model were trials=1, model=tree and winnow=FALSE.
>fit.svmlinear
Support Vector Machines with Linear Kernel
49 samples
168 predictors
2 classes: ‘no’, ‘yes’
Resampling: Cross-Validated (10 fold, repeated 10 times)
Summary of sample sizes: 44, 44, 44, 44, 45, 43, . . .
Resampling results:

### ROC Sens Spec

1 0.96 1
Tuning parameter ‘C’ was held constant at a value of 1
>fit.svmradial
Support Vector Machines with Radial Basis Function Kernel
49 samples
168 predictors
2 classes: ‘no’, ‘yes’
Pre-processing: centered (168), scaled (168)
Resampling: Cross-Validated (10 fold, repeated 10 times)
Summary of sample sizes: 44, 43, 45, 44, 44, 44,
Resampling results across tuning parameters:

| C | ROC | Sens | Spec |
|---|---|---|---|
| 0.25 | 0.9977778 | 0.9883333 | 0.9533333 |
| 0.50 | 1.0000000 | 0.9816667 | 0.9933333 |
| 1.00 | 1.0000000 | 0.9866667 | 0.9933333 |

Tuning parameter ‘sigma’ was held constant at a value of 0.00438827
ROC was used to select the optimal model using the largest value.
The final values used for the model were sigma=0.00438827 and C=0.5.

Example of time series data set used for analysis (4 patient samples, Table 2). Raw data show decrease of NADH at 340 nm, 37° C. Monitoring time in this example is every 5 min for 30 min.

TABLE 2
Exemplary raw measurement data

| Well | 0 Min | 5 Min | 10 Min | 15 Min | 20 Min | 25 Min | 30 Min |
|---|---|---|---|---|---|---|---|
| A1 | 1.26857 | 1.14825 | 1.00028 | 0.850122 | 0.700256 | 0.554552 | 0.417307 |
| A2 | 1.33039 | 1.25651 | 1.19512 | 1.14865 | 1.11217 | 1.08596 | 1.06602 |
| A3 | 1.27575 | 1.12478 | 0.958741 | 0.785704 | 0.61579 | 0.452555 | 0.310368 |
| A4 | 1.27277 | 1.22358 | 1.18633 | 1.15094 | 1.11961 | 1.08782 | 1.05755 |
| A5 | 1.31892 | 1.27451 | 1.25127 | 1.23522 | 1.21413 | 1.19761 | 1.18474 |
| A6 | 1.33023 | 1.26994 | 1.21552 | 1.16176 | 1.10694 | 1.05226 | 0.999643 |
| A7 | 1.26071 | 1.21396 | 1.18168 | 1.15074 | 1.12631 | 1.10273 | 1.07921 |
| A8 | 1.31283 | 1.27415 | 1.253 | 1.23497 | 1.22182 | 1.21158 | 1.20167 |
| A9 | 1.32218 | 1.2434 | 1.17291 | 1.1041 | 0.919813 | 0.966311 | 0.894534 |
| A10 | 1.2504 | 1.18829 | 1.12402 | 1.07154 | 1.00902 | 0.956888 | 0.902667 |
| A11 | 1.31601 | 1.27018 | 1.23352 | 1.21266 | 1.41455 | 1.17203 | 1.15766 |
| A12 | 1.33882 | 1.24255 | 1.34939 | 1.0645 | 0.977156 | 0.886928 | 0.805678 |
| B1 | 1.27768 | 1.15963 | 1.02283 | 0.880338 | 0.738349 | 0.599386 | 0.463488 |
| B2 | 1.33228 | 1.26557 | 1.20652 | 1.16085 | 1.12725 | 1.09873 | 1.07739 |
| B3 | 1.33684 | 1.19362 | 1.04199 | 0.877884 | 0.713342 | 0.553416 | 0.406442 |
| B4 | 1.27572 | 1.23432 | 1.20056 | 1.16667 | 1.13666 | 1.09487 | 1.07808 |
| B5 | 1.32848 | 1.29218 | 1.26804 | 1.24347 | 1.23083 | 1.20873 | 1.20041 |
| B6 | 1.34887 | 1.27915 | 1.23564 | 1.18053 | 1.12951 | 1.08048 | 1.02312 |
| B7 | 1.26282 | 1.21983 | 1.19549 | 1.1692 | 1.1433 | 1.12261 | 1.09833 |
| B8 | 1.31983 | 1.28809 | 1.26538 | 1.24907 | 1.23416 | 1.22408 | 1.21408 |
| B9 | 1.33135 | 1.26073 | 1.19649 | 1.12895 | 1.06186 | 0.997245 | 0.931714 |
| B10 | 1.27897 | 1.21421 | 1.15557 | 1.09822 | 1.04149 | 0.986908 | 0.934232 |
| B11 | 1.32625 | 1.27903 | 1.24407 | 1.21842 | 1.196 | 1.17735 | 1.1619 |
| B12 | 1.35323 | 1.25852 | 1.17454 | 1.09038 | 1.00244 | 0.920261 | 0.838866 |
| C1 | 1.25726 | 1.15913 | 1.04161 | 0.924069 | 0.80692 | 0.684793 | 0.570215 |
| C2 | 1.30206 | 1.25648 | 1.27994 | 1.16624 | 1.1336 | 1.10319 | 1.08187 |
| C3 | 1.34976 | 1.2059 | 1.07721 | 0.940067 | 0.799821 | 0.659655 | 0.525451 |
| C4 | 1.26492 | 1.21156 | 1.16686 | 1.11685 | 1.07117 | 1.02658 | 0.983142 |
| C5 | 1.31633 | 1.2804 | 1.25308 | 1.22862 | 1.20936 | 1.19034 | 1.17503 |
| C6 | 1.3395 | 1.27563 | 1.22613 | 1.1786 | 1.12375 | 1.07 | 1.01772 |
| C7 | 1.25793 | 1.20839 | 1.16656 | 1.12215 | 1.0805 | 1.03971 | 0.999224 |
| C8 | 1.3103 | 1.27283 | 1.25384 | 1.23156 | 1.21864 | 1.20582 | 1.19533 |
| C9 | 1.32655 | 1.25886 | 1.20374 | 1.14134 | 1.07723 | 1.01518 | 0.954185 |
| C10 | 1.26671 | 1.20615 | 1.15426 | 1.09922 | 1.05138 | 1.00763 | 0.965319 |
| C11 | 1.29699 | 1.25674 | 1.234 | 1.21491 | 1.20335 | 1.19244 | 1.18279 |
| C12 | 1.32521 | 1.24689 | 1.17899 | 1.10967 | 1.04321 | 0.977535 | 0.914886 |
| D1 | 1.25007 | 1.1454 | 1.02967 | 0.909196 | 0.787937 | 0.668458 | 0.552616 |
| D2 | 1.29446 | 1.24116 | 1.19385 | 1.15413 | 1.11984 | 1.09117 | 1.06756 |
| D3 | 1.30873 | 1.18908 | 1.0612 | 0.926379 | 0.789862 | 0.652003 | 0.519386 |
| D4 | 1.24969 | 1.19794 | 1.15215 | 1.10665 | 1.06054 | 1.01541 | 0.972247 |
| D5 | 1.30613 | 1.25894 | 1.24667 | 1.22364 | 1.20532 | 1.18796 | 1.17213 |
| D6 | 1.32609 | 1.26598 | 1.21863 | 1.1715 | 1.11845 | 1.06776 | 1.01773 |
| D7 | 1.24413 | 1.19641 | 1.15582 | 1.11233 | 1.07075 | 1.02989 | 0.990333 |
| D8 | 1.29458 | 1.2689 | 1.24792 | 1.23129 | 1.11565 | 1.2037 | 1.18615 |
| D9 | 1.30569 | 1.24427 | 1.1843 | 1.12814 | 1.06402 | 1.00757 | 0.945874 |
| D10 | 1.22895 | 1.17977 | 1.12721 | 1.08437 | 1.05594 | 0.996782 | 0.955109 |
| D11 | 1.28585 | 1.25152 | 1.24202 | 1.20863 | 1.19789 | 1.18557 | 1.17687 |
| D12 | 1.32485 | 1.25974 | 1.19634 | 1.12607 | 1.06529 | 1.00369 | 0.945298 |
| E1 | 1.26585 | 1.24468 | 1.22993 | 1.21788 | 1.20687 | 1.19663 | 1.18684 |
| E2 | 1.28588 | 1.26609 | 1.25555 | 1.24634 | 1.23834 | 1.23301 | 1.22454 |
| E3 | 1.40492 | 1.38842 | 1.38142 | 1.37631 | 1.37537 | 1.35759 | 1.37925 |
| E4 | 1.25224 | 1.22717 | 1.22456 | 1.20899 | 1.20537 | 1.19215 | 1.18729 |
| E5 | 1.28445 | 1.26099 | 1.2578 | 1.24481 | 1.24227 | 1.24312 | 1.22902 |
| E6 | 1.41559 | 1.39233 | 1.39125 | 1.39526 | 1.39198 | 1.39374 | 1.39444 |
| E7 | 1.24941 | 1.23203 | 1.22008 | 1.21025 | 1.19981 | 1.19116 | 1.18131 |
| E8 | 1.28294 | 1.26681 | 1.25575 | 1.24786 | 1.23931 | 1.23234 | 1.22482 |
| E9 | 1.41624 | 1.40295 | 1.39325 | 1.38915 | 1.38831 | 1.39135 | 1.3926 |
| E10 | 1.25103 | 1.2344 | 1.22255 | 1.21066 | 1.19934 | 1.19019 | 1.18001 |
| E11 | 1.28282 | 1.26508 | 1.25373 | 1.2456 | 1.23579 | 1.22951 | 1.22388 |
| E12 | 1.40091 | 1.38208 | 1.37353 | 1.36838 | 1.37118 | 1.37277 | 1.37596 |
| F1 | 1.27381 | 1.25177 | 1.42954 | 1.22591 | 1.2172 | 1.20462 | 1.19653 |
| F2 | 1.31582 | 1.27527 | 1.26896 | 1.2579 | 1.25167 | 1.24271 | 1.23626 |
| F3 | 1.40752 | 1.38937 | 1.38345 | 1.3758 | 1.37474 | 1.37291 | 1.38097 |
| F4 | 1.25509 | 1.23478 | 1.2266 | 1.21557 | 1.20815 | 1.19841 | 1.19044 |
| F5 | 1.2965 | 1.27722 | 1.26885 | 1.26017 | 1.25426 | 1.24492 | 1.23656 |
| F6 | 1.4187 | 1.39706 | 1.3931 | 1.38531 | 1.38785 | 1.38914 | 1.39011 |
| F7 | 1.246 | 1.22802 | 1.21812 | 1.20745 | 1.19826 | 1.18993 | 1.18121 |
| F8 | 1.29022 | 1.27465 | 1.26567 | 1.25686 | 1.24998 | 1.24217 | 1.23517 |
| F9 | 1.41724 | 1.40326 | 1.39596 | 1.38903 | 1.3893 | 1.39166 | 1.39336 |
| F10 | 1.24859 | 1.23024 | 1.21949 | 1.2079 | 1.19759 | 1.18777 | 1.17869 |
| F11 | 1.28758 | 1.26826 | 1.25715 | 1.24684 | 1.23775 | 1.23027 | 1.22347 |
| F12 | 1.40227 | 1.38389 | 1.37645 | 1.26949 | 1.37169 | 1.35972 | 1.37408 |
| G1 | 1.18531 | 0.919044 | 0.594516 | 0.246662 | 0.142402 | 0.141582 | 0.141228 |
| G2 | 1.20175 | 1.05315 | 1.02611 | 1.01948 | 1.01648 | 1.01242 | 1.00878 |
| G3 | 1.28761 | 1.03063 | 0.803991 | 0.588096 | 0.414563 | 0.292175 | 0.216463 |
| G4 | 1.1742 | 0.931209 | 0.636465 | 0.316307 | 0.142279 | 0.141106 | 0.140634 |
| G5 | 1.21033 | 1.06008 | 1.02962 | 1.02425 | 1.01994 | 1.01586 | 1.01182 |
| G6 | 1.30585 | 1.05157 | 0.817872 | 0.603655 | 0.429518 | 0.306902 | 0.229231 |
| G7 | 1.16608 | 0.935107 | 0.650304 | 0.339653 | 0.13371 | 0.141104 | 0.140561 |
| G8 | 1.19612 | 1.05326 | 1.01648 | 1.01461 | 1.00731 | 1.00632 | 1.00034 |
| G9 | 1.30101 | 1.0522 | 0.814083 | 0.599029 | 0.430415 | 0.298879 | 0.22278 |
| G10 | 1.1673 | 0.931729 | 0.659445 | 0.348536 | 0.142251 | 0.140762 | 0.140366 |
| G11 | 1.21504 | 1.05768 | 1.02159 | 1.01397 | 1.00866 | 1.00364 | 1.00117 |
| G12 | 1.32031 | 1.04353 | 0.796514 | 0.578628 | 0.406124 | 0.287374 | 0.216896 |
| H1 | 1.17455 | 0.890913 | 0.547969 | 0.195446 | 0.139742 | 0.139161 | 0.138134 |
| H2 | 1.19457 | 1.04327 | 1.0205 | 0.996873 | 1.01074 | 0.998359 | 1.00137 |
| H3 | 1.31764 | 1.06892 | 0.873249 | 0.682162 | 0.525871 | 0.402686 | 0.312871 |
| H4 | 1.15428 | 0.892204 | 0.579256 | 0.244908 | 0.138164 | 0.13817 | 0.137432 |
| H5 | 1.19279 | 1.04071 | 1.01799 | 1.01421 | 1.0075 | 1.00471 | 0.998634 |
| H6 | 1.30429 | 1.03489 | 0.79438 | 0.582402 | 0.415275 | 0.296579 | 0.222946 |
| H7 | 1.16315 | 0.909108 | 0.599414 | 0.271546 | 0.139156 | 0.138371 | 0.138153 |
| H8 | 1.1959 | 1.0441 | 1.01703 | 1.01247 | 1.00661 | 1.00317 | 0.998108 |
| H9 | 1.31983 | 1.04303 | 0.800813 | 0.586715 | 0.416342 | 0.302321 | 0.229748 |
| H10 | 1.16614 | 0.9116 | 0.605442 | 0.280762 | 0.138635 | 0.137896 | 0.13746 |
| H11 | 1.20422 | 1.04782 | 1.0182 | 1.01127 | 1.00568 | 1.00119 | 0.996086 |
| H12 | 1.31099 | 1.01021 | 0.753462 | 0.526417 | 0.355479 | 0.246476 | 0.189229 |

### EXAMPLE 7: RISK CLASSIFICATION OF CHRONIC LYMPHOCYTIC LEUKEMIA AND COLON CANCER PATIENTS BY MONITORING ENZYME KINETICS

### 7.1 Methods

Overview: PBMCs (peripheral blood mononuclear cells) from chronic lymphocytic leukemia (CLL) patients and cancer tissue from the primary tumor of colorectal cancer (CRC) patients were analyzed with the EnFin®-test kits in anaerobic tissue/blood culture for activity of key metabolic enzymes responsible for anaerobic adaptation. Kinetic data including linear and non-linear enzyme activities were vectorized with all single time series data piled up to a single vector. Popular Machine Learning algorithms including SVM, RF, C5.0 and Neural Networks were trained on the enzyme kinetic datasets generated with the enzymatic test kits and evaluated using separated datasets.

Specific description: For CLL the study sample consisted of 22 patients diagnosed with CLL who presented at the University Hospital Heidelberg between 2013 and 2014 and were treated with CIT. Peripheral blood mononuclear cells (PBMCs) were isolated by Ficoll gradient. The research was approved by the Ethics Committee of the University of Heidelberg (S-356/2013 and S-254/2016).

For CRC the study sample consisted of 101 patients diagnosed with CRC who presented at the University Hospital Heidelberg between 2009 and 2012 and were depicted from the DACHS study (German Cancer Research Center) within the framework of the IMPACT consortium (“Improving long-term prognosis and quality of life of patients with colorectal cancer”. Analysis was done with cryo-frozen tissue samples from the primary tumor available through the clinical research unit (KFO 227) of the University Hospital Heidelberg, consistent quality was granted by the tissue biobank of the National Center for Tumor Diseases (NCT) in Heidelberg, Germany. The research was approved by the Ethics Committee of the University of Heidelberg (KFO 310/2001).

### Genetic Aberrations and CEA Serum Levels

Chromosomal aberrations by fluorescence in situ hybridization (FISH) as well as TP53 and Immunoglobulin Heavy Chain Variable (IGHV) mutation status were obtained from medical reports. Preoperative Carcinoembryonic Antigen (CEA) serum levels were obtained from medical reports and were available for 99 colorectal cancer patients. Sera were obtained on the day before surgery.

### Assay Software Part: Benchmark Methods and Performance Evaluation

Experiments were run in an Intel Core Duo 2 T6600, 2.2 GHz, and 3 GB of RAM under Windows 7 environment. The algorithms were coded in R (R 3.5.1 (www.R-project.org)) using caret, gtools, openxlsx, reshape2, c1071, plyr, DMwR, randomForest, nnct and C50 packages. We selected well-known learning algorithms (Support Vector Machine (SVM), RF, C5.0) to be used in conjunction with a popular method for handling imbalanced data. All algorithms were used both with and without in combination with Synthetic Minority Oversampling Technique (SMOTE) technique. SMOTE was applied to alter the number of instances, such that the amount of instances in each class became more balanced. To achieve this, SMOTE combines the features of existing instances with the features of their nearest neighbors to create additional synthetic instances. K-Nearest Neighbor (KNN) was set default (=5) (Wang et al. (2015), Comput Methods Programs Biomed 119 (2): 63-76; Alghamdi et al. (2017), PLoS One 12 (7): e0179805.) Data was splitted in training and testing sets (70% and 30% respectively). Due to the small sample size of available patients, we performed repeated k-fold cross validation (RkCV) to avoid overfitting. K-fold cross validation splits the training data into k sets of equal size, and uses k−1 sets to train and predict the remaining set. For each one of the subsets, RkCV performs k-fold cross validation for several times with k random splits of the training data. As implementation, we used the R package caret with its trainControl settings defined to perform “repeatedcv”, 10 repeats, 10 folds and the smote sampling parameter to resolve class imbalances resulting from the splitting. To train we used a grid search to tune the hyper-parameters of the classification algorithm while performing cross validation. For each of the evaluated algorithms (SVMLinear, SVMRadial, RF, C5.0 and two neural networks (avNNet and pcaNNet)), the model was used to predict the classification on the testing set and the following performance measurements were recorded: accuracy, sensitivity, specificity, positive and negative predictive values. To assure that results were robust, the whole process was repeated 5 times for random training/test partitions of the initial data.

To evaluate the effectiveness we compared accuracy results of the algorithms. Additionally we reported sensitivity, specificity, positive predictive value, negative predictive value, recall, F1 (F-measure), prevalence, detection prevalence, detection rate and balanced accuracy. Accuracy was calculated as follows:

Accuracy=(TP+TN)/(TP+TN+FP+FN) 1.

where TP denotes true positives, TN denotes true negatives, FP denotes false positives, and FN denotes false negatives. To define the diagnostic sensitivity and specificity the following equations were used: For CLL: Sensitivity [%]=100×(number of high risk patients (defined as progress within 2 years after CIT)/total number of high risk patients. Specificity [%]=100×(number of low risk patients (defined as no progress event (within 2 years) after CIT)/total number of low risk patients. For CRC: Sensitivity [%]=100×(number of high risk patients (defined as recurrent within 2 years after surgery)/total number of high risk patients. Specificity [%]=100×(number of low risk patients (defined as not recurrent within 2 years after surgery)/total number of low risk patients.

### Assay Hardware Part: Sample Preparation and Analysis

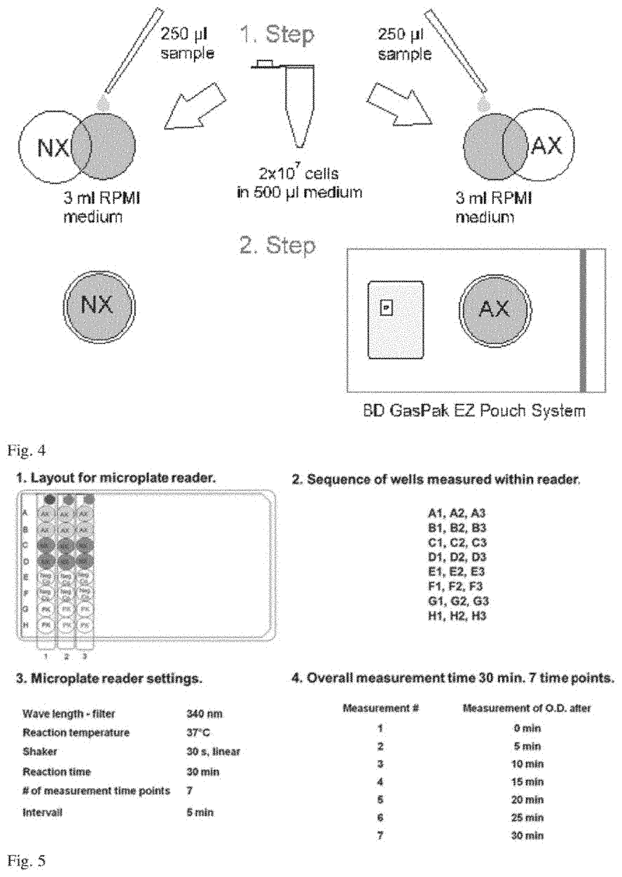

Sample preparation and analysis was performed with EnFin-CLL-Test™ kits (CE IVD, #6102 ENF, EnFin® GmbH, Germany) and EnFin-CRC-Test™ kit s (RUO, #980010-6101 ENF) for CLL and CRC respectively according to the instructions of the manufacturer. Briefly, two 3 cm petri dishes per patient were filled with 3 ml RPMI 1640 (Life Technologies, Paisely, UK) and 1*10⁷ cells (CLL) or fragmented (25 mg) primary tumor tissue (CRC). For CLL and CRC respectively, one was wrapped with an oxygen impermeable shell (GasPak™ EZ, Becton Dickinson, New Jersey, USA) to generate anoxic (=anaerobic) conditions. After incubation (for CLL samples: 16-24 h, for cryo CRC samples: 5 h) at 37° C. and 5% CO 2 (anoxic (Ax) and normoxic (Nx) sample), cells were washed and resolved in 500 μl of the provided buffer solution (FIG. 4). Enzymes were extracted by ultrasound (Diagenode Bioruptor® Sonication System, Diagenode, Seraing, Belgium), 1 μg protein per well was loaded following a fixed pipetting protocol (FIG. 5) ensuring correct data import later by the software. Activities of PK-la (low affinity), PK-ha (high affinity) and LDH were monitored as decrease of NADH at 340 nm for 30 min at 37° C. in a microplate reader (VICTOR X 2030, Perkin Elmer, Waltham, USA), run in duplicates, comprising the whole range of enzyme activity, i.e. linear and non-linear kinetics. Positive and negative controls were within the recommended ranges.

### Statistical Analyses

Statistical analyses were performed using statistical software R 3.5.1 (www.R-project.org). Endpoints were defined according to iwCLL criteria or ESMO (European Society for Medical Oncology) guidelines. Patients' characteristics in the two groups defined by clinical high risk versus low risk were compared by Fisher's Exact test for categorical parameters and t-tests or Mann-Whitney-tests for metric parameters. Accuracy estimates and 95% confidence intervals (CI) were calculated, and lower limits of the CI >50 are interpreted as superiority over chance.

### 7.2 Results

Overview: CLL patients harboring anaerobic cells in their samples relapsed very early after chemo-immunotherapy (CIT), However, recommended clinical markers in CLL, TP53 and IGHV mutation analysis, failed in predicting response to CIT. In CRC both CEA high serum levels (above 2.5 ng/ml) and presence of anaerobic cells predicted early recurrence after surgery with curative intention. Machine learning outperformed all markers in both cancers. The best results showed an accuracy of 99% (95% CI 98-100) for CLL and an accuracy of 91% (95% CI 88-93) for colorectal cancer.

### Specific Description: CLL and CRC Test Cohorts

96 chronic lymphocytic patients were prospectively enrolled in our trial, of these 74 were eligible to be analyzed with the EnFin-CLL™ test kits. With the kits 27 cases were classified as High Risk (anaerobic growth) and 47 as Low Risk (aerobic growth). Both risk groups showed similar clinical parameters including cytogenetic abnormalities, TP53 mutation and lymphocyte doubling time. Clinical characteristics are shown in Table 3.

TABLE 3
Patient characteristics CLL.

| | Kit HR | | Kit LR | | |
|---|---|---|---|---|---|
| | (n = 27) | | (n = 47) | | p-value |
| Kit, median (range) | 1.59 | (1.24-3.28) | 1.91 | (1.70-2.26) | 0.071 |
| Age at diagnosis, median | 63.9 | (40-81.9) | 63.9 | (31.8-83.8) | 0.63 |
| (range) [years] | | | | | |
| Age ≥65 years, n (%) | 13 | (48) | 22 | (48) | 1 |
| Age ≥75 years, n (%) | 4 | (15) | 4 | (9) | 0.457 |
| Gender female/male | 12/15 | | 18/29 | | 0.631 |
| WBC median (range) [/nl] | 57290 | (20570, 247200) | 61640 | (17200, 234100) | 0.823 |
| PB lymphocytes, median | 94 | (82, 99) | 90 | (67, 100) | 0.089 |
| (range) [%] | | | | | |
| BRAF mut n (%) | 0 | (0) | 3 | (8) | 0.281 |
| MYD88 mut n (%) | 0 | (0) | 1 | (3) | 1 |
| NOTCH1 mut n (%) | 3 | (13) | 3 | (8) | 0.666 |
| SF3B1 mut n (%) | 4 | (17) | 3 | (8) | 0.408 |
| del11q22-23 n (%) | 2 | (8) | 8 | (18) | 0.303 |
| trisomy 12 n (%) | 1 | (4) | 12 | (27) | 0.023 |
| del13q14 n (%) | 20 | (77) | 26 | (58) | 1 |
| del17p13 and/or TP53mut | 6 | (25) | 8 | (22) | 0.765 |

Patients were classified as clinical high risk (CLL-HR) when having progressive disease within two years after beginning of CIT, as low risk (CLL-LR) when having no progressive disease within two years after beginning of CIT. CIT included bendamustine in combination with rituximab (BR), cyclophosphamide/doxorubicine/vincristine/prednisolone in combination with rituximab (R-CHOP), chlorambucil in combination with rituximab (R-CBL) or obinutuzumab (G-CBL) and fludarabine/cyclophosphamide in combination with ofatumumab (O-FC). 22 patients were treated with CIT. This dataset was used for machine learning. The ratio of CLL-HR to CLL-LR in the dataset was balanced with 1:1.2.

Out of the 101 colorectal cancer patients from the DACHS study 22 were classified as high risk (recurrent within two years after surgery). This dataset was used for machine learning. The ratio of CRC-HR to CRC-LR in the dataset was unbalanced with 1:4.6. Clinical characteristics are shown in Table 4.

TABLE 4
Patient characteristics CRC.

| | HR (n = 22) | | LR (n = 79) | | p-value |
|---|---|---|---|---|---|
| Kit, mean, sd (range) | 1.78, 0.67 | (0.09-3.16) | 2.15, 0.80 | (0.08-5.08) | 0.049 |
| Age at diagnosis, mean, sd (range) | 65.6, 10 | (46-90) | 62.5, 11.1 | (33-81) | 0.243 |
| [years] | | | | | |
| Age ≥65 years, n (%) | 12 | (55) | 38 | (48) | 0.636 |
| Age ≥75 years, n (%) | 3 | (14) | 8 | (10) | 0.701 |
| Gender female | 9 | (%) | 25 | (%) | 0.451 |
| CEA, median (range) [/nl] | 4.2 | (0.8-390) | 1.8 | (0.2-1334.7) | 0.04 |
| Adj. Chemotherapy n (%) | 18 | (82) | 38 | (49) | 0.007 |
| CA19-9, median (range) | 22.5 | (2.6-95905.2) | 11.1 | (0.5-2432.8) | 0.032 |
| BRAF mut n (%) | 2/11 | (18)* | 0/27 | (0)* | 0.49 |
| Kras mut n (%) | 6/11 | (55)* | 5/27 | (19)* | 0.061 |
| MSI n (%) | 0/10 | (0)* | 2/22 | (9)* | 1 |

| | | | |
|---|---|---|---|
| Localization rectum/colon (%) | 71/29 | 60/40 | 0.324 |
| Margin R0/R1 (%) | 67/33 | 92/8 | 0.002 |
| UICC stage 1/2/3/4 (%) | 6/13/25/56 | 14/42/38/6 | <0.0005 |
| Radiotherapy (%)^{†} | 43 | 35 | 0.138 |

Notably, both risk groups consisted of colon and rectal cancer with a dominance of rectum cancer patients (for high risk 71% rectum cancer, for low risk 60%). According to treatment standards, the majority of rectal cancer patients were subjected to radiotherapy in both groups. Both risk groups showed similar mutational characteristics regarding Ras, BRAF, and MSI. Importantly, 82% of the patients in the high risk group received adjuvant chemotherapy compared to 49% in the low risk group (Table 2).

### Data Preprocessing and Feature Space

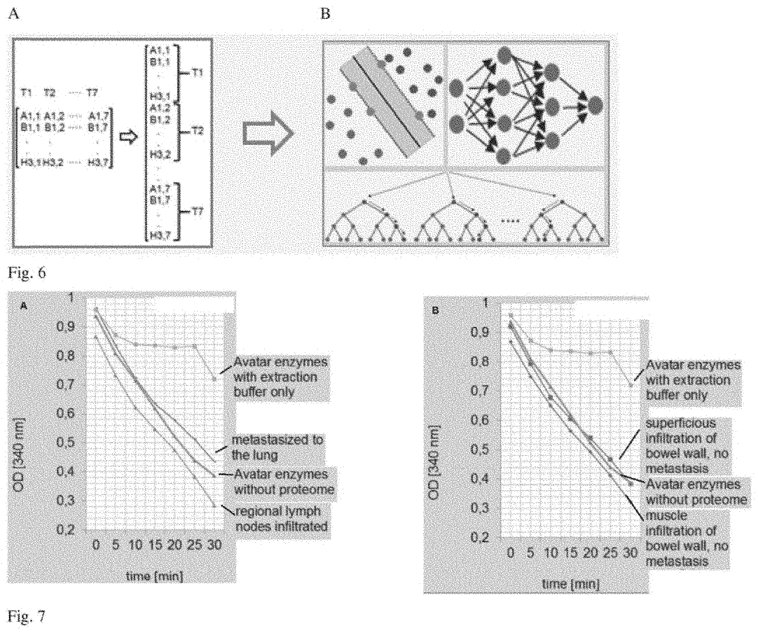

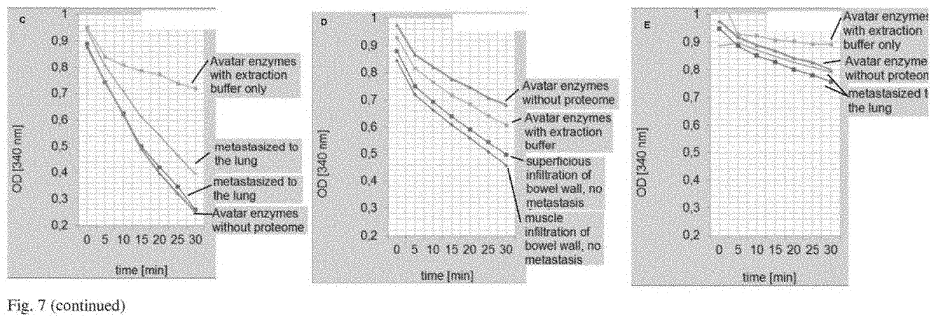

For correct labeling as high risk (=“1”) or low risk (=“0”) for the supervised training of the machine learning algorithms unsorted raw data from the microplate reader were sorted by patients as shown in FIG. 6. The code is disclosed in the GitHub repository www.github.com/enfinlab. Using the whole range of enzyme activity (substrate (S) to enzyme (E) ratio=S<<E; S≈E and S>>E), i.e. including enzymatic activity within the non-linear range under two conditions (with oxygen and without oxygen as described above) we did vectorization of the time series, so creating a single vector with all single time series data piled up to a single vector (FIG. 6). In this most simple approach for each binary classification problem (CLL/CRC) the feature space consisted of 168 features respectively. Thus all time series points, positive and negative controls (noise) and both conditions (aerobic/anaerobic) are each represented in a single feature.

### AI Classifier Outperform Both Gold-Standard Biomarkers TP53, IGHV and CEA and the EnFin® Assay

CLL patients with TP53 aberrations and IGHV unmutated status respond poorly to CIT. A very simple feed-forward neural network (pcaNNet) with one neuron in the hidden layer outperformed these standard clinical markers (TP53 mutation analysis and IGHV mutation analysis) in the original CLL dataset by far (no significant results for TP53 and IGHV, Table 3). PcaNNet was as accurate as the kit assay (detecting anaerobic leukemia cells in the patients PBMC samples) in finding CLL-HR cases, however, it outperformed slightly the kit regarding positive/negative predictive values, that are in particular important for clinical decision making (pcaNNet: PPV=90%-98%, NPV=64%-100%; EnFin-CLL-Test™, kit: PPV=100%, NPV=50%, Tables 5, 6). In addition synthetically oversampled (SMOTEd) datasets with the same data distribution as in the original dataset were created to test algorithm performance on a larger sample size. In the synthetically oversampled CLL dataset all algorithms showed near perfect (mean accuracy results-99%, Table 5) classification results.

TABLE 5
Machine learning performance, CLL dataset (n = 22): first value: without SMOTE, second value:
SMOTE within 10 × 3 cross-validation resampling procedure, third value: SMOTEd CLL dataset.
All machine learning results are mean values from 5 runs with unseen test datasets (hold-out sets).

| | | | Dataset | | | | |
|---|---|---|---|---|---|---|---|
| | | | CLL | | | | |
| | | | Accuracy | | | | |
| Metrics | | | (95% CI) | SE | SP | PPV | NPV |
| Algorithm | SVMLinear | Support | 53 (48-58); | 47; 53; | 60; 67; 98 | 52; 83, 98 | 60; 57; 100 |
| and | | Vector | 60* (55-65); | 100 | | | |
| Algorithm | | Machine | 99* (98-100) | | | | |
| Family | SVM Radial | | 50 (45-55); | 20; 67; | 80; 60; 98 | NaN; NaN; | 50; 70; 100 |
| | | | 63* (58-68); | 100 | | 98 | |
| | | | 99* (98-100) | | | | |
| | RF | Bagging | 53 (46-58); | 47; 73; | 60; 53; 98 | 52; 52; 98 | 60; 81; 100 |
| | | | 63* (58-68); | 100 | | | |
| | | | 99* (98-100) | | | | |
| | C5.0 | Boosting | 73* (69-78); | 73; 73; | 73; 53; 98 | 75; 63; 98 | 82; NaN; 97 |
| | | | 63* (58-68); | 96 | | | |
| | | | 97* (96-99) | | | | |
| | avNNet | Neural | 57* (52-62); | 47; 53; | 67; 80; 97 | 48; 80; 96 | 61; 62; 97 |
| | | Network | 67* (62-71); | 96 | | | |
| | | | 96* (94-98) | | | | |
| | pcaNNet | | 70* (85-75); | 47; 53; | 93; 80; 98 | 90; 83; 98 | 64; 62; 100 |
| | | | 67* (62-72); | 100 | | | |
| | | | 99* (98-100) | | | | |
| Standard | TP53 | | 25 | 18 | 100 | 100 | 10 |
| | IGVH | | 71 | 80 | 50 | 80 | 50 |

| | | | | | |
|---|---|---|---|---|---|
| Kit | 69 | 55 | 100 | 100 | 50 |

P-values: IGHV, p = 0.52; del17p, p = 0.64; Kit-CLL, p = 0.037; CEA, p = 0.027; Kit-CRC, p = 0.049
SE = Sensitivity; SP = Specificity; PPV = Positive Predictive Value; NPV = Negative Predictive Value,
*superior over chance.

TABLE 6
Machine learning performance, CRC dataset (n = 101): first value: SMO1E within
10 × 10 cross-validation resampling procedure, second value: SMOTEd CRC dataset. All machine
learning results are mean values from 5 runs with unseen test datasets (hold-out sets).

| | | | Dataset | | | | |
|---|---|---|---|---|---|---|---|
| | | | CRC | | | | |
| | | | Accuracy | | | | |
| Metrics | | | (95% CI) | SE | SP | PPV | HPV |
| Algorithm end | SVMLinear | Support | 60* (56-64); | 23; 100 | 70; 98 | 14; 98 | 78; 100 |
| Algorithm | | Vector | 90* (88-93) | | | | |
| Family | SVM Radial | Machine | 76* (72-79); | 10; 85 | 93; 92 | NaN; 90 | 80; 100 |
| | | | 89* (87-92) | | | | |
| | RF | Bagging | 67* (63-71); | 27; 89 | 77; 85 | 25; 82 | 80; 92 |
| | | | 87* (84-90) | | | | |
| | C5.0 | Boosting | 65* (61-69); | 23; 85 | 76; 98 | 18; 84 | 79; 89 |
| | | | 87* (84-90) | | | | |
| | avNNet | Neural | 62* (58-66); | 33; 96 | 70; 85 | 23; 83 | 80; 97 |
| | | Network | 90* (89-94) | | | | |
| | pcaNNet | | 68* (64-72), | 37; 97 | 77; 86 | 32; 84 | 82; 100 |
| | | | 91* (88-93) | | | | |
| Standard | CEA | | 66 | 61 | 50 | 22 | 85 |

| | | | | | |
|---|---|---|---|---|---|
| Kit | 69 | 18 | 84 | 24 | 79 |

P-values: IGHV, p = 0.52; del17p, p = 0.64; Kit-CLL, p = 0.037; CEA, p = 0.027; Kit-CRC, p = 0.049.
SE = Sensitivity; SP = Specificity; PPV = Positive Predictive Value; NPV = Negative Predictive Value,
*superior over chance.

It is well known that CEA is very inconsistent in detecting recurrence in CRC. Nevertheless there is no alternative, both approved and more reliable, biomarker, thus CEA in combination with other surveillance measures is still gold standard in clinics for recurrence detection. However, it is not used for predicting Disease-Free-Survival (DFS). Predicting DFS in CRC with a test as early as possible, e.g. at the time point of surgery, would be very beneficial for follow-up and treatment of patients. In particular, it is important to detect patients recurrent after curative surgery (resection of the primary tumor plus metastasis if present) within two years after surgery as several studies have shown that their tumors are very aggressive. These patients could benefit from aggressive management integrating chemotherapy/radiation plus alternative targeted drugs and intensive follow-up. In this study we compared the classification performance of CEA, the EnFin-CRC-Test™ and AI algorithms (that were trained with the raw data from the EnFin-CRC-Test™) in predicting DFS after curative intended surgery. The EnFin-CRC-Test™ detecting anaerobic cells in the CRC tissue and preoperative elevated CEA serum levels (threshold 2.5 ng/ml) showed similar performance with accuracies of 69% or 66%, a PPV of 24% or 22% and a NPV of 79% or 85% (Table 6). Neural networks (pcaNNet) had a comparable accuracy (68%, Table 6), however, they showed better predictive value performance (PPV=32%; NPV=84%, Table 6) that is critical for decision making in clinical routine. In the synthetically oversampled CRC dataset all algorithms showed excellent classification results (mean accuracy results ranging from 87%-91%, Table 6). Here pcaNNet reached best results (91% accuracy, Table 6).

Taken together AI algorithms were superior to clinically approved or recommended/experimental biomarkers. Very simple neural networks (pcaNNet) with default settings²⁸ had the overall best performance. They showed both superior performance on unseen real-life clinical data and near perfect classification results on unseen synthetically oversampled datasets (from real-life datasets used in this study). Although the low size of samples and default settings of ML algorithms used, we have shown that it is possible to learn from enzyme kinetic data and outperform extensively validated clinical markers that have been used for a long time for clinical decision making.

### EXAMPLE 8: MODULATION OF ENZYME ACTIVITY BY EXTRACTS FROM FORMALIN-FIXED AND EMBEDDED CELLS (“FFPE PROTEOME”)

20 FFPE tissue sections (10 μm) of 10 colon carcinomas (2 sections per patient) with different clinical courses (UICC stages I-IV, I: superficial infiltration, II: infiltration of the muscle layer of the colon, III: infiltration of regional lymph nodes, IV: distant metastases) were used. Approximately 500 μg of proteome were extracted, 4 μg thereof were added to the reaction volume. In particular PKLA shows differences in its activities. Overall there is a total of 168 data points (feature space) per tumor. From the complex relationships of the data points, depending on the control activities present at each measurement point (activities with extraction buffer or without extraction buffer (and without proteome, respectively)), in particular machine learning can search for patterns that correlate with the disease progress/individual treatment.

### NON-STANDARD LITERATURE CITED

Alghamdi et al. (2017), PLoS One 12 (7): e0179805
Alves-Filho et al. (2016), Front. Immunol.; doi: 10.3389/fimmu.2016.00145
Amoedo et al. (2013), Biosci. Rep. 33:c00080
Arts et al., (2017), J. Leukoc. Biol. 101:151
Cascone et al. (2018), Cell Metabolism 27: 977
Cheng et al. (20169, Nat Immunol., 17 (4):406
Delano ct al. (2016), JCI 26:23
EP2821790 A1
Gatenby et al. (2004), Nature Reviews Cancer 4:891
Gdynia et al. (2018), EBioMedicine 32:125
Irving et al. (2017), Front. Immunol. doi: 10.3389/fimmu.2017.00267
Kojima et al. (2017) Nature Chemical Biology, doi:10.1038/nchembio.2498
Lee et al. (20189, PNAS 115 (19):E4463
Odegaard et al. (2013), Immunity 38:644
Palsson-McDermott et al. (2015), Cell Metabolism 21:65;
Pearce et al. (2013), Immunity 38 (4):633
Roybal et al. (2017), Annu. Rev. Immunol. 35:229
Venereau et al. (2012), J. Exp. Med. 209 (9):1519
Wang et al. (2015), Comput Methods Programs Biomed 119 (2): 63-76.
Wasmuth et al. (2005), Journal of Hepatology 42:195
Wein et al. (2010), Stem Cell Res 4:129
WO2017/098051 A2
WO 2018/108327

1. A method of determining a metabolic adaptation of a living entity of interest to a first set of environmental conditions and to a second set of environmental conditions comprising
(a) determining with a first substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₁ and t₂ after starting the determining reaction;
(b) determining with a second substrate concentration at least two activities of at least one enzyme comprised in a specimen of said living entity maintained under said first set of environmental conditions and at least two activities of said at least one enzyme comprised in a specimen of said living entity maintained under said second set of environmental conditions, wherein said activities are determined at two non-identical points in time t₃ and t₄ after starting the determining reaction; wherein said second substrate concentration is at most twofold, preferably is about equal to or lower than, the K_{M} of said enzyme for said substrate; and
(c) determining the metabolic adaptation of said living entity based on comparing at least one non-linear activity determined in step (a) and/or (b) to at least one further activity determined in step (a) and/or (b).

2. The method of claim 1, wherein said first substrate concentration (i) is at least twofold, preferably at least fivefold, more preferably at least tenfold the K_{M} of said enzyme for said substrate; or (ii) wherein said first substrate concentration is at most twofold, preferably is about equal to or lower than, the K_{M} of said enzyme for said substrate and is non-identical to the second substrate concentration.

3. The method of claim 1, wherein at least step (c) is computer-implemented, preferably by training an automated machine learning algorithm with the data of steps (a) and (b) of cells having a known metabolic adaptation.

4. The method of claim 1, wherein said first environmental condition is normoxia and wherein said second environmental condition is hypoxia and wherein said metabolic adaptation is switch of energy metabolism from oxidative phosphorylation under normoxia to glycolysis under hypoxia.

5. The method of claim 1, wherein said at least one enzyme is pyruvate kinase, preferably pyruvate kinase M2.

6. The method of claim 5, wherein said substrate is pyruvate and wherein said first substrate concentration is 10 mM and wherein said second substrate concentration is 0.1 mM.

7. The method of claim 5, wherein a strong change in the activity of either high-affinity pyruvate kinase (PKHA) or low-affinity pyruvate kinase (PKLA) under hypoxic conditions as compared to the activity under normoxic conditions is indicative of a successful switch from oxidative phosphorylation under normoxia to glycolysis under hypoxia, and/or wherein a moderate or no change in the activity of either PKHA or PKLA under hypoxic conditions as compared to the activity under normoxic conditions, or a parallel change of both PKHA and PKLA, is indicative of an unsuccessful switch from oxidative phosphorylation under normoxia to glycolysis under hypoxia.

8. The method of claim 1, wherein at least one of said activities determined in steps (a) and (b) is a non-linear activity.

9. (canceled)

10. A method for determining an activation status of immune cells in a test sample comprising said immune cells, comprising
(a) incubating a first subportion of said test sample comprising immune cells under normoxic conditions,
(b) incubating a second subportion of said test sample comprising immune cells under hypoxic conditions,
(c) determining the activities of at least the enzymes high-affinity Pyruvate Kinase (PKHA) and low-affinity Pyruvate Kinase (PKLA) in cells of said first and second subportions,
(d) comparing said activities determined in step (c), and
(e) based on the result of comparison step (d), determining the activation status of the immune cells in said test sample.

11. (canceled)

12. The method of claim 10, wherein said immune cells are peripheral blood mononuclear cells (PBMCs), preferably are T-cells or hematopoietic stem cells, more preferably are CD34+ hematopoietic stem cells.

13. (canceled)

14. A method of determining a modulation of at least one enzyme activity by an extract of a fixed cell sample and optionally providing a risk classification for a patient suffering from disease, comprising
(i) providing at least a first and a second aliquot of said at least one enzyme;
(ii) contacting said second aliquot with said extract of a fixed cell sample;
(iii) determining the activity of the first aliquot of step (i) and the activity of the second aliquot of step (ii);
(iv) comparing the activities of the first aliquot and the second aliquot determined in step (iii), and thereby
(v) determining a modulation of at least one enzyme activity by an extract of a fixed cell sample.

15. The method of claim 14, wherein said fixed cell sample is a sample of a subject.

16. The method of claim 15, wherein said determining step (iii) further comprises
(I) determining with a first substrate concentration at least two activities of said first aliquot and at least two activities of said second aliquot, wherein said activities are determined at two non-identical points in time t₁ and t₂ after starting the determining reaction; and
(II) determining with a second substrate concentration at least two activities of first aliquot and at least two activities of said second aliquot, wherein said activities are determined at two non-identical points in time t₃ and t₄ after starting the determining reaction; wherein said second substrate concentration is at most twofold, preferably is about equal to or lower than, the K_{M} of said enzyme for said substrate.

17. The method of claim 14, wherein said fixed cell sample is an aldehyde-fixed cell sample, preferably a formaldehyde- and/or glutaraldehyde-fixed sample, preferably a formaldehyde-fixed sample.

18. (canceled)

19. (canceled)

20. (canceled)


SEQUENCE LISTING (5 sequences)

SEQ ID NO: 1 - protein, 15 aa, Artificial
  P-M2tide
  MOD_RES (8)..(8): phosphotyrosine
GGAVDDDYAQFANGG

SEQ ID NO: 2 - protein, 75 aa, Homo sapiens
KFKDPNAPKRPPSAFFLFCSEYRPKIKGEHPGLSIGDVAKKLGEMWNNTAADDKQPYEKKAAKLKEKYEKDIAAY

SEQ ID NO: 3 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 4 - protein, 215 aa, Artificial
  Gln-HMGB1
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEQRPKIKGEHPGLSIGDVAKKLGEMWNNTAADDKQPQEKKAAKLKEKQEKDIAAQRAKGKPDAAKKGVVKAEKSKKKKEEEEDEEDEEDEEEEEDEEDEDEEEDDDDE

SEQ ID NO: 5 - protein, 215 aa, Artificial
  Glu-HMBG1
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEERPKIKGEHPGLSIGDVAKKLGEMWNNTAADDKQPEEKKAAKLKEKEEKDIAAERAKGKPDAAKKGVVKAEKSKKKKEEEEDEEDEEDEEEEEDEEDEDEEEDDDDE